(19) United States
(12) Patent Application Publication
(10) Pub. No.: US 2026/0260767 A1
(43) Pub. Date: Sep. 3, 2026

(54) SYSTEM AND METHOD FOR HEALTHCARE CONTENT GENERATION

(71) Applicant: Radiant Graph, Inc., San Francisco, CA (US)

(72) Inventors: Amin Mousavi, San Francisco, CA (US); Anmol Madan, San Francisco, CA (US); Robert James Ellis, San Francisco, CA (US); Priyanshi Jain, San Francisco, CA (US); Sai Moturu, San Francisco, CA (US)

(73) Assignee: Radiant Graph, Inc., San Francisco, CA (US)

(21) Appl. No.: 19/446,716

(22) Filed: Jan. 12, 2026

Related U.S. Application Data
(60) Provisional application No. 63/744,040, filed on Jan. 10, 2025.

(51) Int. Cl. G16H 50/70; G16H 70/60

(52) U.S. Cl. CPC G16H 50/70; G16H 70/60

(57) ABSTRACT

In variants, the method can include determining a dataset for a set of members, optionally processing the dataset, optionally determining a set of features based on the dataset, determining a set of cohorts including members from the set of members, and generating content for a cohort.

### CROSS-REFERENCE TO RELATED APPLICATIONS

This application claims the benefit of US Provisional Application number 63/744,040 filed 10-JAN-2025, which is incorporated in its entirety by this reference.

### TECHNICAL FIELD

This invention relates generally to the healthcare analytics field, and more specifically to a new and useful system and method in the healthcare analytics field.

### BRIEF DESCRIPTION OF THE DRAWINGS

FIG. 1 is a schematic representation of a variant of the method.

FIG. 2 is a schematic representation of an example of the method.

FIG. 3 is a schematic representation of an example of the method.

FIG. 4A is a schematic representation of an example of a propensity model.

FIG. 4B is a schematic representation of an example of tuning predictive models.

FIG. 4C is a schematic representation of an example of generating cohorts using a latent space of a predictive model.

FIG. 4D is a schematic representation of an example of a propensity model.

FIG. 5 is a schematic representation of an example of generating content.

FIG. 6 is a schematic representation of an example of the method.

FIG. 7A is an illustrative example of ranked features (e.g., ranked selected features).

FIG. 7B is an illustrative example of cohorts.

FIG. 7C is an illustrative example of an analysis of cohorts.

FIG. 8A is a schematic representation of an example of training model(s) for content generation.

FIG. 8B is a schematic representation of an example of content generation.

FIG. 9 is a schematic representation of a specific example of content generation.

FIG. 10A is a schematic representation of a specific example of processing data.

FIG. 10B is a schematic representation of a specific example of the method.

FIG. 11 is a schematic representation of a specific example of the system (e.g., a platform).

FIG. 12 is an illustrative example of generated content.

FIGS. 13A and 13B are illustrative examples of a user interface

FIGS. 14A and 14B are illustrative examples of statistical analyses used for feature selection.

FIGS. 15A-15E are illustrative examples of ranked features (e.g., ranked selected features).

FIGS. 16A and 16B are illustrative examples of cohorts.

FIG. 17 is an illustrative example of feature values for a set of members.

FIG. 18 is an illustrative example of determining cohorts using a ‘waterfall’ method.

FIG. 19 is an illustrative example of a variant of a dashboard user interface.

FIG. 20 is an illustrative example of a variant of a strategy user interface.

FIGS. 21A-21B are an illustrative example of a variant of a user interface.

### DETAILED DESCRIPTION OF THE INVENTION

The following description of the embodiments of the invention is not intended to limit the invention to these embodiments, but rather to enable any person skilled in the art to make and use this invention.

### 1. Overview

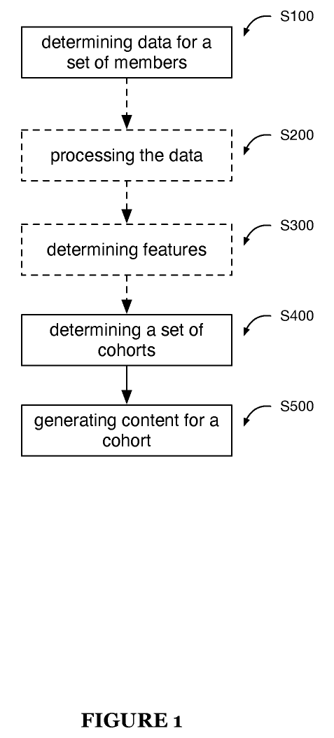

As shown in FIG. 1, variants of the method can include: determining datasets for each of a set of members S100; optionally processing the data S200; determining a set of features S300; determining a set of cohorts S400; optionally generating content strategy S420; and generating content for a cohort S500. In variants, the system can include: a data engine 100; a propensity model 200; a cohort generator 300; an optional smart choice model; a cohort description model 400; an optional strategy generator 500; a content generator 600; a user interface 700; and a campaign executor. The system and method function to generate personalized content based on health-related information.

In an illustrative example, the method includes: receiving a set of data associated with a set of members; extracting a first set of features for each member in the set of members; determining a set of cohort selection features from the first set of features; determining a set of cohorts based on the set of cohort selection features; generating a set of metrics for each cohort in the set of cohorts; optionally, based on the set of metrics, generating a content strategy for a cohort in the set of cohorts; based on the set of metrics for the cohort and/or the content strategy, generating content for the cohort. However, the system / method can be otherwise performed.

### 2. Technical advantages

Variants of the technology can confer one or more advantages over conventional technologies.

In personalized care, health care decisions rely on the individual's unique health conditions, constraints, health goals, and desired outcomes. Past studies suggest that adopting a personalized approach to healthcare can significantly reduce the use of medical services and lower costs. The studies also indicate that involving patients more in their care planning is not only ethically correct but also cost-effective. This care approach can enable providers to tend to patients not only medically but also ethically, emotionally, mentally, socially, and financially. This strategy has various benefits, such as: improved outcomes by enabling communication and coordination of personalized patient care, improved satisfaction levels among patients and their families, and patient involvement in their care planning. The growing demand for personalized care is hindered by manual processes, which are further complicated by workforce shortages, competing priorities, and inadequate training. These factors collectively impede the effective implementation of this approach to care. The process of developing personalized communications and/or a personalized care plan is laborious, which makes it extremely difficult to implement on a large scale.

First, variants of the technology can provide a platform designed for healthcare organizations to implement large-scale personalization and drive member (e.g., patient) engagement. In an example, variants of the technology can determine member cohorts, targeted for a specific use case, with human-readable descriptors that can be used for personalizing content for individual cohorts. In a first specific example, variants of the technology can determine personalized communications for a cohort (e.g., to engage members). In a second specific example, variants of the technology can determine a personalized care plan for a cohort (e.g., determining benefits and/or programs that are more likely to positively impact the members in the cohort). In a third specific example, variants of the technology can determine a predicted value (e.g., a propensity score) for a set of content delivery methods for a member and/or a cohort of members. The content and parameters of the content delivery can be modified to optimize for cost efficiency, conversion, or any other goal. In a second example, variants of the technology can reduce the context length of the model input by determining a set of features and/or derived metrics from a set of member data and generating a strategy and/or content based on the features and/or metrics. In a third example, variants of the technology can implement large scale personalization while complying with regulations for privacy and safety. In a first specific example, variants of the technology can determine content according to a set of guardrails that control which content can be included in healthcare communication. The guardrails can be enforced as absolute rules in the model inputs (e.g., in the prompts), by secondary models that check the generated content against the guardrails, within or in conjunction with large language model (LLM)-based systems, including orchestration frameworks or architectures (e.g., retrieval-augmented generation, LangChain, agent-based systems, etc.), by manual reviewers, and/or otherwise enforced. In a second specific example, variants of the technology can generate content while streamlining the human review process, such as by aggregating human-reviewed assets. In a third specific example, variants of the technology can protect member privacy by generating custom content for a cohort of members using metrics associated with the cohort, rather than the member.

Second, variants of the technology can enrich incomplete healthcare data, which can enable improved personalized content generation despite the complexities of healthcare data at scale. In a first example, the data can be enriched by correlating member attributes (e.g., location) to social determinants of health data, which can provide signals that can be leveraged in cohort selection and content generation. In a second example, the data can be enriched by inferring incomplete data. In an illustrative example, missing gender labels can be inferred based on correlations between gender labels and known data.

Third, variants of the technology can customize communication with a member while maintaining their privacy. In an example, individual member's data can be processed to extract a member feature set; member cohorts can be determined based on the member feature set; and customized content can be generated based on features of the cohort, rather than based on the individual member.

However, further advantages can be provided by the system and method disclosed herein.

### 3. System

In variants, the system can include: a data engine 100; a propensity model 200; a cohort generator 300; an optional smart choice model; a cohort description model 400; an optional strategy generator 500; a content generator 600; an user interface 700; and a campaign executor. The system functions to generate customized content for cohorts of members.

The system can run locally or on a remote computing system (e.g., cloud system). The system can include different instances for different entities; alternatively, the same instance can be shared across entities.

The data engine 100 functions to determine a member feature set for each member in the set of members. The features are preferably semantic, but can alternatively be nonsemantic. The features are preferably human readable, but can alternatively be not human readable. The features are preferably user-provided, but can alternatively be dynamically generated (e.g., by the data engine, etc.), be looked up (e.g., be a set of statistical measures, be a set of predetermined code classes, be a set of reimbursement code hierarchical levels, etc.), and/or be otherwise determined.

Each feature can be associated with a set of candidate values. The set of candidate values for a feature can be continuous, discrete, binary, discretized, and/or otherwise continuous or discontinuous. The set of candidate values can be qualitative, quantitative, and/or any other type of values.

The feature set can include demographic features (e.g., metabolic panels, thyroid panels, cardiac biomarkers, etc.), behavioral traits (e.g., smoking, alcohol use, drug use, etc.), diagnostic markers (e.g., HbA1c levels, lipid profiles, metabolic panels, thyroid panels, and cardiac biomarkers), claims data (e.g., ICD codes, NDC codes, CPT codes, etc.), clinical data (e.g., inpatient, outpatient, pharmacy claims, etc.), Social Determinants of Health (SD0H) Metrics (e.g., income level, educational attainment, employment status, housing safety, food insecurity, population density associated with the member's zip code, etc.), communication preferences, sentiment (e.g., from call transcripts), product and/or service (e.g., app, web portal, device, clinical services, surveys, etc.) usage, marketing interactions (e.g., clicks, opens, responses, registration, engagement, churn, etc.), and/or any other features.

A feature set is preferably extracted for each member, but can alternatively be extracted for a set of members.

The extracted feature set can be sparse (e.g., lack values for some or most features), dense (e.g., include values for most features), complete (e.g., include values for all features), and/or have any other density. Values for different features can be extracted for different members; alternatively the values for the same features can be extracted for different members. Different values for a given feature can be extracted for different members; alternatively the same value for a given feature can be extracted. In an example, the data engine can extract the feature values of "ICD code = Z79.4" and "HbA1c = 7.0%" from the data set for a given member.

The data engine 100 preferably includes a Large Language Model (LLM), but can alternatively include a set of rules (e.g., take the first 3 characters of an ICD code), programmed process, a parametric model, statistical models (e.g., K-means clustering, gaussian mixture models, etc.), classifiers, logistic regressors, random forest models, neural networks (e.g., feedforward MLPs, etc.), support vector machines, and/or any other type of model.

The data set that the features are extracted from for a given member can be received from the member, received from the user (e.g., a healthcare organization, etc.), received from health records (e.g., electronic health records), scraped from social media, historical content campaigns (e.g., marketing campaigns, engagement campaigns, etc.; from the user or from other entities), third parties (e.g., AHRQ dataset, census data, presidential election results, RUCA codes, PLACES / SVI datasets, etc.), and/or any other data set. In an example of receiving data from a third party, the data can be purchased.

The data set can include claims data (e.g., ICD codes, NDC codes, CPT codes, etc.), clinical data (e.g., inpatient, outpatient, pharmacy claims, etc.), conversion information (e.g., whether the user took a given action when content was presented to in a given format, etc.), call center data, member settings, product usage, service usage, marketing interactions, health data, member preference data, member-reported intent data, and/or any other data.

In variants, the data engine 100 can optionally include a feature processor and a feature selector.

The feature processor functions to preprocess the data and/or features. The feature processor can include one or more data processes, including deduplicating, cleaning, inferring, imputing, normalizing format, handling missing fields, handling invalid entries, denoising, and/or any other data processes. The feature processor can include rule-based models, probabilistic record-linkage models, graph based entity resolution models, statistical outlier models, unsupervised anomaly detectors, imputation models (e.g., k nearest neighbors, etc.), deterministic transforms, encoding models, named entity recognition models, other parsing models, and/or other processor.

However, the feature processor may be otherwise configured.

The feature selector can reduce the overall context length of the content-generation model input. Reducing the overall context length of the content-generation model input can be particularly helpful because healthcare datasets (e.g., claims data) contain tens of thousands of potential features (e.g., code numbers), but few datapoints. This can cause the input vector with values for each feature to be extremely long, even though most of the features will be valueless (e.g., null). The feature selector can determine the correlation between each feature and the target outcome, a set of features and the target outcome, and/or any other correlations. The feature selector is preferably determined based on feature data for members paired with historical target outcomes (e.g., what the user is interested in achieving), but can alternatively be determined based on other information. The selected features are preferably determined based on all feature data for all members, alternatively based only on feature data from members that have been tested for the target outcome, and/or based on any other member data. The selected features are preferably determined based on population data, alternatively based on data for a single member. The feature selector can be a statistical test (e.g., chi-squared test, proportion test, etc.), but can alternatively include a deterministic decision tree; a neural network; a set of rules; a programmed process; and/or any other test or model. The feature selector can be deterministic or stochastic. The number of features can be between 1-100,000 or any range or value therebetween (e.g., at least 2, at least 10; at least 100; at least 10,000; etc.), but can alternatively be greater than 100,000.

The feature selector can optionally convert the feature value for each member into a binary value (e.g., the feature is valued / not valued for the member; the feature value is above or below a threshold; etc.), optionally convert the target outcome to a binary value (e.g., successful / not), compute the correlation between each feature and the target outcome (e.g., whether the feature has a strong positive, strong negative, or other correlation with the target outcome; computing a p-value; etc.), rank or score the feature based on the correlation, and/or filter for the strongest features (e.g., strongest positive, strongest negative, etc.; using a statistical threshold or cutoff, etc.). In an example, converting the feature value to a binary value can use fuzzy mapping (e.g., to distinguish between a 0 value and null). The strongest features are used to determine the set of cohorts, determine the cohort description, determine the cohort metrics fed into the content generator, and/or otherwise used.

However, other feature selection and/or dimensionality reduction methods (e.g., mutual information, distance correlation, recursive feature elimination, tree-based importance, etc.) can be used to subselect the features.

However, the feature selector may be otherwise configured.

However, the data engine 100 may be otherwise configured.

The propensity model 200 functions to predict the likelihood of a member to take an action. The propensity model 200 can preferably generate predictions per member, but can alternatively generate predictions per goal, per cohort, per delivery mechanism, and/or any other prediction target. The propensity model 200 can preferably receive a member feature set from the data engine, but can additionally or alternatively receive a content strategy from the strategy generator, cohort features, cohort metrics, member data, a set of guardrails, a target variable, a goal, a set of product data, a set of engagement data, resource constraints (e.g., channel capacity), a predetermined set of candidate content, and/or any other data. The propensity model 200 can be a decision tree, linear model (e.g., logistic regression, etc.), tree-based model, neural network, recommender-system architecture, causal uplift model, and/or other model architectures. The propensity model 200 can be learned (e.g., from historic members that had the opportunity to take the action), and/or otherwise determined. The propensity model can be learned based on the member's feature value set, the action value (e.g., which piece of content was presented to the member, which outreach channel was used, etc.), the engagement value (e.g., whether the member took the action), and/or other information. The propensity score can be determined based on: the member's feature value set; the candidate action value (e.g., the candidate content, the candidate outreach channel, etc.), and/or other information. The propensity model 200 can determine the propensity score for historic members, prospective members (e.g., members who have not had the opportunity to take the action), and/or for any other set of members.

The predicted member actions can include retention (e.g., cancel, renew, downgrade, become inactive, etc.), conversion (e.g., enroll, subscribe, upgrade, sign up, purchase, switch plans, add dependents, etc.), engagement (e.g., use a service, log in, engage with content, complete onboarding, download an app, join a program, respond to care reminders, respond to nudges, enroll in disease management programs, etc.), financial behavior (e.g., pay on time, default, accept a payment plan, be price-sensitive, set up autopay, appeal a claim, etc.), care utilization (e.g., schedule an appointment, use preventive services, seek urgent care, seek an emergency visit, use telehealth, delay care, forgo care, etc.), treatment adherence (e.g., fill a prescription, refill medications on time, adhere to a plan, switch to generics, discontinue medication, etc.), and/or any other predicted member actions.

Determining the propensity score can include associating a predicted value with the member action, including costs, expected values of a member action, and/or any other associations. The costs can include cost per content generated, cost per delivery, cost per conversion, cost per avoided adverse outcome, and/or latency costs (e.g., reduced effectiveness due to delayed delivery). The expected values of a member action (e.g., net value, absolute counts, probability weighted values, etc.) can include incremental value of a member action, cost avoidance, utilization impact (e.g., change in visit frequency, medication adherence rate, care intensity, etc.), lifetime value (LTV) impact, churn reduction, and/or risk score change (e.g., clinical risk, actuarial risk, or predicted adverse events, etc.).

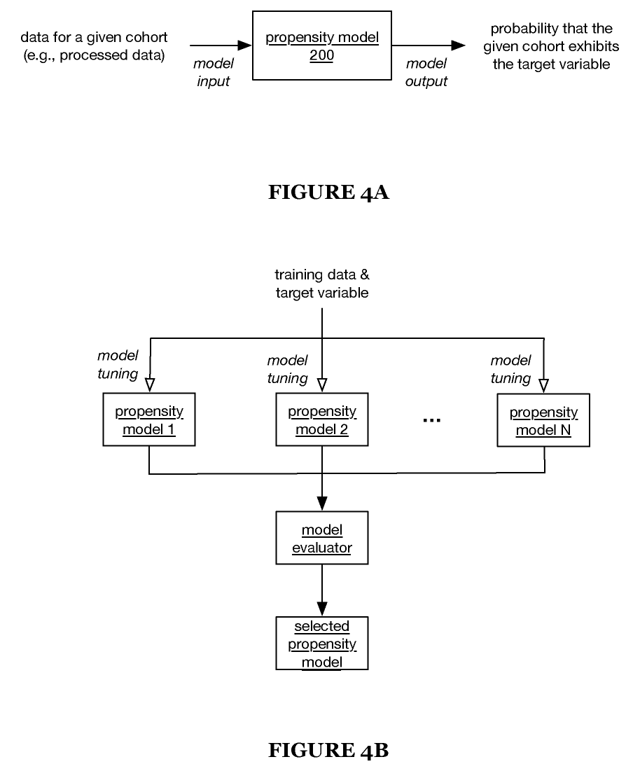

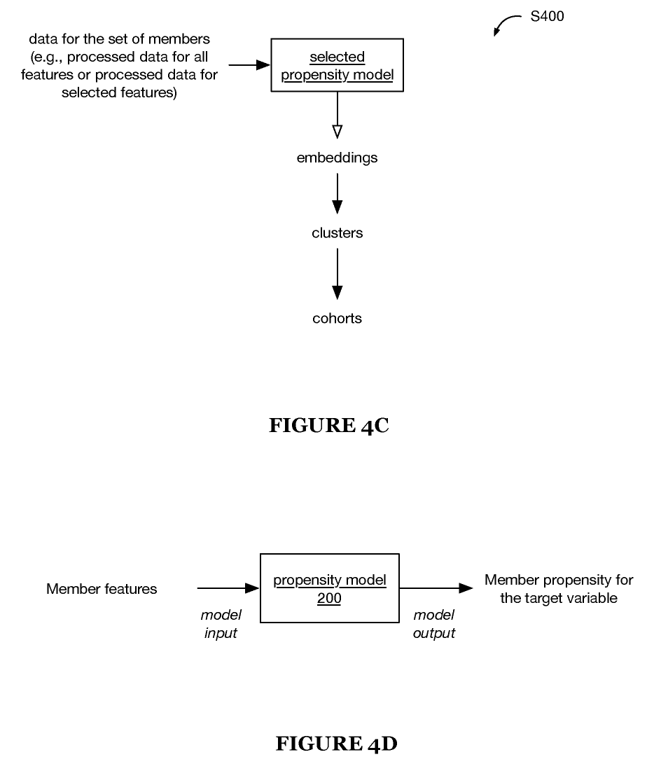

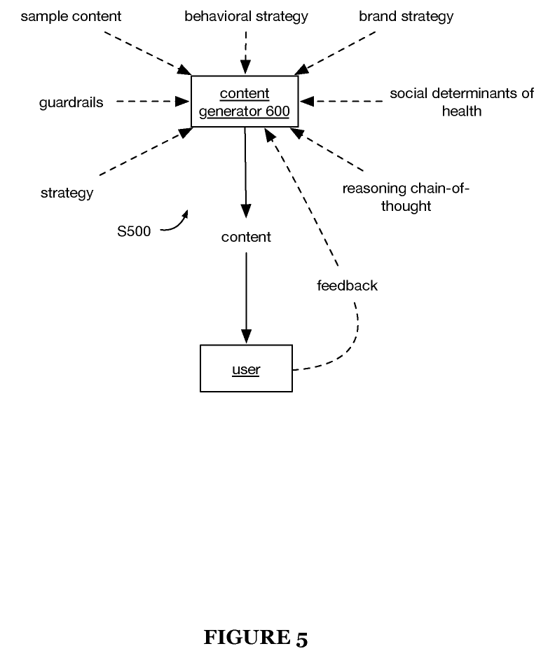

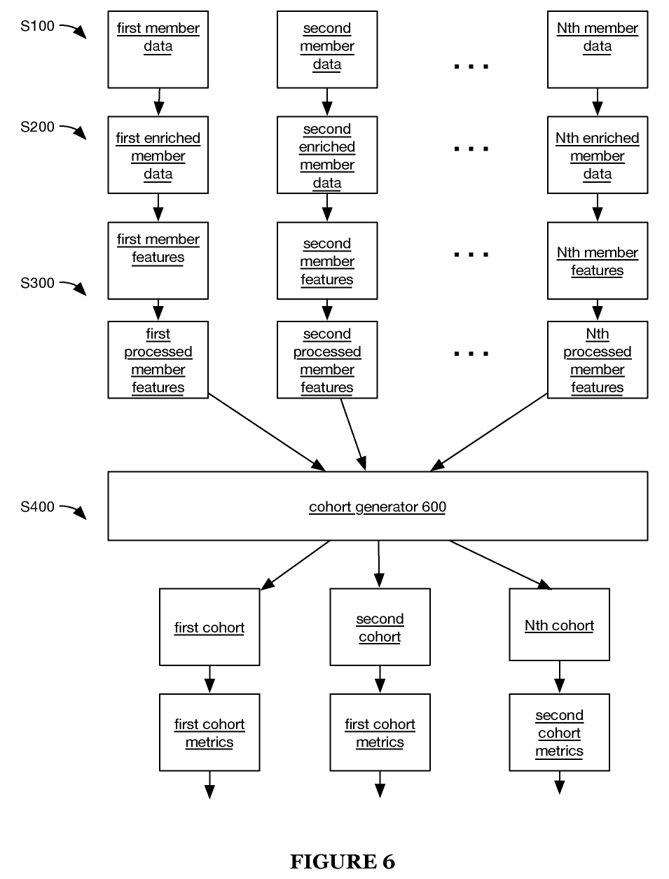

An example of the propensity model 200 is shown in FIG. 4D.

In a first variant, the propensity score for a member can be used to determine which members should receive given outreach (e.g., mailers, calls, etc.). In an example of the first variant, members with propensity scores for mailers above a threshold can receive a mailer, while those with propensity scores below the threshold do not receive a mailer. In an example, the propensity score for a member can be used to determine which members should receive high-cost outreach (e.g., "Smart Spend").

In a second variant, the propensity score for the members in a cohort can be used to determine which outreach methods should be used for the cohort outreach strategy. In an example, outreach methods with low overall propensity scores can be excluded from the strategy, while outreach methods with high overall propensity scores can be included as part of the strategy.

However, the propensity model 200 can be otherwise configured.

The cohort generator 300 functions to generate cohorts of members. The resultant cohorts are preferably interpretable (e.g., human-readable), but can alternatively be not human readable. The resultant cohorts are preferably deterministic, but can alternatively be probabilistic. The resultant cohorts are preferably mutually exclusive or distinct (e.g., do not include overlapping members), but can alternatively overlap or be otherwise related. The resultant cohorts preferably collectively cover all or most of the member population (e.g., 99%. 90%, 80%, 70%, 60%, etc.), but can alternatively cover a majority of the member population, a minority of the member population, and/or any other subset of the member population.

The cohorts are preferably generated from the feature sets for the member population, or more preferably from the filtered feature sets, but can alternatively be generated from the unfiltered feature set, the propensity scores, and/or any other information. The cohorts can additionally or alternatively be generated based on population-level features extracted from the feature sets, user inputs (e.g., natural language prompts, user selected thresholds, user selected feature permutations, etc.), a preferred number of cohorts (e.g., as specified by the user), and/or other information. The generated cohorts can be adjusted (e.g., by a user, by a set of rules, etc.), but can alternatively not be adjusted.

Each cohort is preferably defined by a combination of feature values (e.g., features and corresponding values), but can alternatively be defined by other criteria, wherein members that satisfy the set of feature values are included in the cohort. In an example, a cohort can be defined by a set of threshold values and/or inclusion values for a set of cohort features (e.g., "HbA1C > 8%" AND "gender = male", where the cohort features are "HbA1C" and "gender", and the values are "> 8%" and "male"). The features can be combined by logical operators (e.g., AND, OR, XOR, etc.) or otherwise combined.

The cohort generator 300 can include a model (e.g., neural network), decision tree, set of rules, and/or any other components.

In a first variant, the cohort generator 300 can include an iterative greedy algorithm that maximizes population coverage. In a first embodiment, the cohort generator can iteratively track members that are already assigned a cohort (e.g., by masking them out, removing them from the pool, ignoring them, etc.), determine a combination of features from the remaining available features (e.g., from the filtered features, etc.) based on ability to cover the unassigned population (e.g., members not assigned to a cohort), assign members satisfying the combination of features to the new cohort, and stop iteratively generating cohorts when a stop condition is met (e.g., threshold number of iterations met, less than a threshold proportion or number of members are unassigned, feature combination length exceeds a threshold, incremental cohort size falls below a threshold, etc.). In a second embodiment, the cohort generator can generate a plurality of different feature value permutations (e.g., from the filtered set of features, from all features, etc.) and select the set of feature value permutations that collectively have the highest coverage of the member population.

In an illustrative example, the resultant cohorts for a member population can include "proactive wellness", "maternal wellness", "behavioral wellness", "diabetes and heart health", "adolescents", and/or "student wellness".

The resultant cohorts can be selected by a user (e.g., for content generation), used to monitor different subsets of the member population (e.g., for engagement, for the target metric, etc.), and/or otherwise used.

In a second variant, the cohort generator 300 can include determining cohorts based on data from the propensity model. In a specific example of the second variant, the cohorts can be generated based on a group of members with the highest expected value for a particular delivery mechanism.

However, the cohort generator 300 may be otherwise configured.

The system can optionally include a smart choice model, which functions to dynamically select specific content and outreach channel permutations for each member and/or cohort. The smart choice model can prioritize actions and next best offers on a per-member basis based on predicted outcomes. The smart choice model preferably determines the permutation based on the feature set for the member, but can alternatively use other information. The content can be dynamically generated for the member (e.g., based on the respective feature set, the content generation inputs, etc.), be selected from a predetermined set of content (e.g., generated for the cohort that the member belongs to, generated for the campaign, etc.); and/or otherwise determined. The outreach channel can be selected from a set of predetermined outreach channels (e.g., mailer, email, call, text, etc.), but can be otherwise determined.

The smart choice model can use reinforcement learning (RL) or bandit approaches (e.g., multi-armed bandit, contextual bandit, etc.) to determine the content and outreach channel permutation for a given member. In an example, the smart choice model can pick a combination of contextual variables and content, then dynamically update its strategy (e.g., policy) based on the reward. In a specific example, if a member finds a service helpful or completes a screening, probabilities update for the entire population; if a member finds the service unhelpful or does not complete the screening, the model is penalized, and/or otherwise adjusted. The smart choice model and/or content can be updated according to member outcomes (e.g., interactions, engagements, product utilization, etc.).

However, the smart choice model may be otherwise configured.

The cohort description model 400 functions to determine descriptions for a cohort. The cohort description model 400 can function to obfuscate or abstract away from individual members, which can minimize the risk of providing clinical advice or leaking private health information. The descriptions can include statistical metrics (e.g., distribution of a given feature's values across the cohort members), cohort definition (e.g., in natural language), cohort insights (e.g., human-readable definition of segment, summary of unique cohort patterns, etc.), outreach hypotheses (e.g., based on the cohort members' propensity scores for different outreach channels, etc.), and/or any other descriptions. Examples of statistical metrics can include cohort size, demographic distribution (e.g., age, gender, etc.), and/or other statistics.

The descriptions can be generated using a statistical model, a natural language model (e.g., LLM), a vision model (e.g., diffusion model), manually generated, and/or otherwise generated. Illustrative examples of description elements that can be generated can include "proactive wellness", "maternal wellness", "behavioral wellness", "diabetes and heart health", "adolescents", "student wellness", and/or any other descriptions. An illustrative example of a description that can be generated can include “'maternal and reproductive health needs for mothers with young children, who are in the age groups of 20-35, have been diagnosed with post-partum issues (depression, anemia, gestational diabetes), have previously not engaged with the service, but have opened a marketing email.”

The cohort description model 400 preferably receives data from the cohort generator (e.g., cohort data, cohort features, member data, etc.), but can alternatively receive data from user input, the data engine, and/or any other data source.

The cohort description model 400 can include a set of cohort selection rules, a statistical model (e.g., to generate descriptive statistical metrics), a value calculator, a model, and/or any other component. The set of cohort selection rules can include feature-based filters, thresholds, ranges, categorical inclusion/exclusion, bins, temporal filters, event-based categories, and/or any other cohort selection rules.

However, the cohort description model 400 may be otherwise configured.

The system can optionally include a strategy generator 500, which functions to generate a content strategy for a cohort in the set of cohorts. The content strategy (e.g., outreach strategy) can be: a decision tree or DAG of outreach steps, and/or otherwise defined. Each outreach step can include a triggering condition, an outreach channel (email, text, mail, video, call, etc.), content, a marketing schedule (e.g., when to follow up, when to terminate the step, when to begin the step, etc.) and/or any other outreach step elements.

The strategy generator 500 can receive the cohort descriptions, data generated by the propensity model, user input from the user interface, feature sets for the members in a cohort, and/or any other data. The strategy generator 500 preferably includes a large language model, but can additionally or alternatively include any other suitable model, deterministic process, decision tree, and/or any other process.

Alternatively, the strategy can be received from a user, retrieved from a historical session (e.g., campaign), and/or otherwise determined.

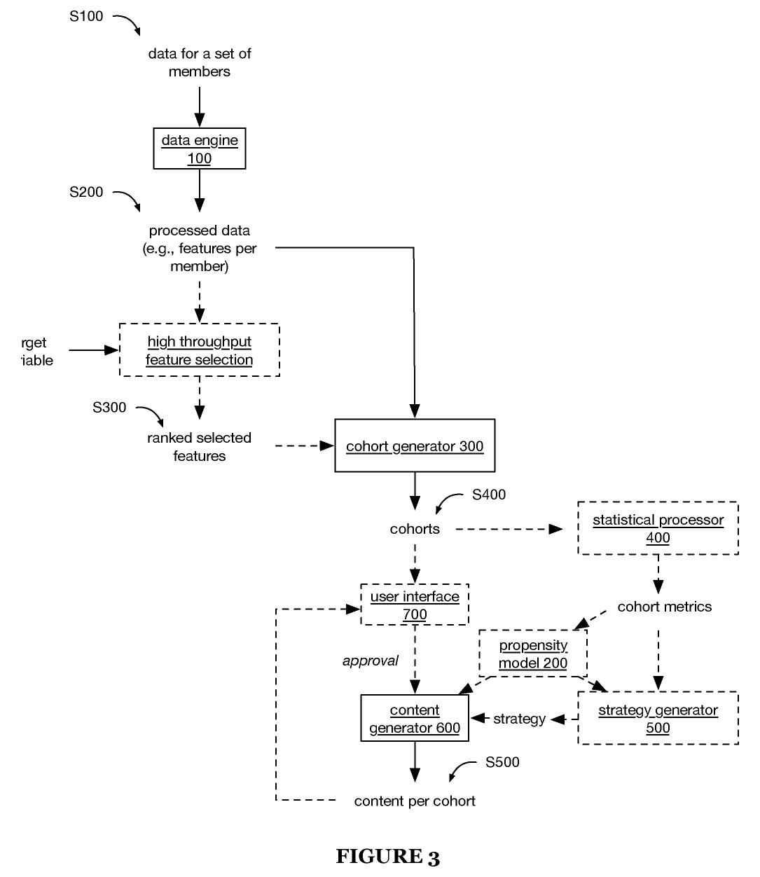

An example of the strategy generator 500 is shown in FIG. 3.

However, the strategy generator 500 may be otherwise configured.

The content generator 600 functions to generate content for a cohort in the set of cohorts. The content can include: text, a script, video, audio, and/or any other modality of content. The content can be personalized to a member, a cohort, and/or other set of members. The content generator 600 can generate the content based on: outputs of the strategy generator, the cohort description(s) for a cohort, user input from the user interface (e.g., user guidance), default guardrails (e.g., rules, conditioning inputs, etc.), and/or any other inputs. In an example, the content generator can generate cohort content based on the cohort's statistical distribution of a set of generation features (e.g., demographic, etc.). The generation features preferably exclude the cohort-defining features (e.g., not the features used to define the cohort, used to cluster the members into the cohort), but can alternatively include the cohort-defining features or be the cohort-defining features (e.g., the distribution of HbA1C values for members in the cohort when the cohort is defined by members with HbA1C > 8%).

The user guidance can include past emails and content to mimic, client brand guidelines, content constraints (e.g., character limits), message goal/intent, campaign/initiative goal, brand voice, emotional register, compliance rules (e.g., rules that must be strictly adhered to, such as no clinical advice or treatment, no medication management decisions, etc.), writing manuals, formatting constraints, text blocks that should not be modified, behavior change strategies (e.g., nudge theory, motivators, etc.), marketing schedule, call to action, and/or any other user guidance. All of the inputs are preferably natural language, but can alternatively be a structured request (e.g., an API call, etc.) and/or otherwise structured.

The content generator 600 can include a generative model (e.g., LLM), but can additionally or alternatively include a set of rules, decision trees, and/or be otherwise structured.

In variants, the system can optionally include a set of critic or guard models that enforce the compliance rules (e.g., default guardrails, user-entered compliance rules, etc.), wherein the content is not sent to the member or to the user for review when the content fails the critic or guard model evaluation. The critic or guard models can include: a set of heuristics, rules, a neural network (e.g., LLM), feature extractor paired with a rule, and/or any other model.

However, the content generator 600 may be otherwise configured.

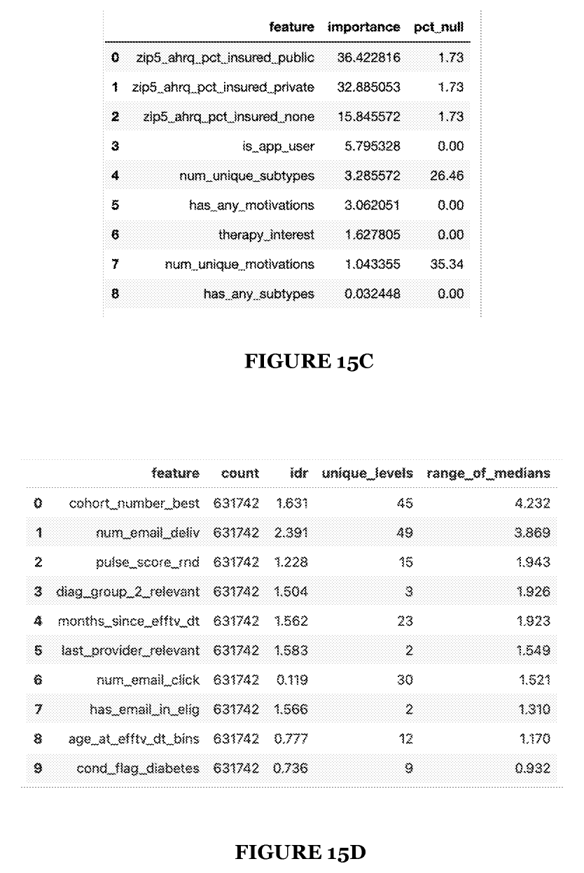

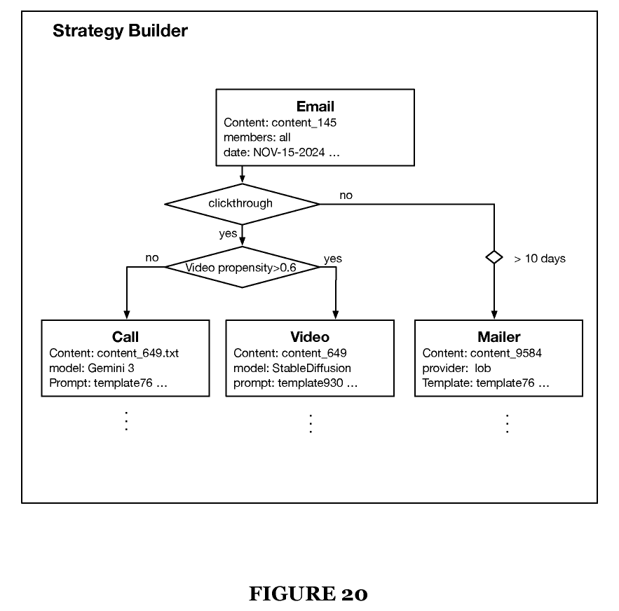

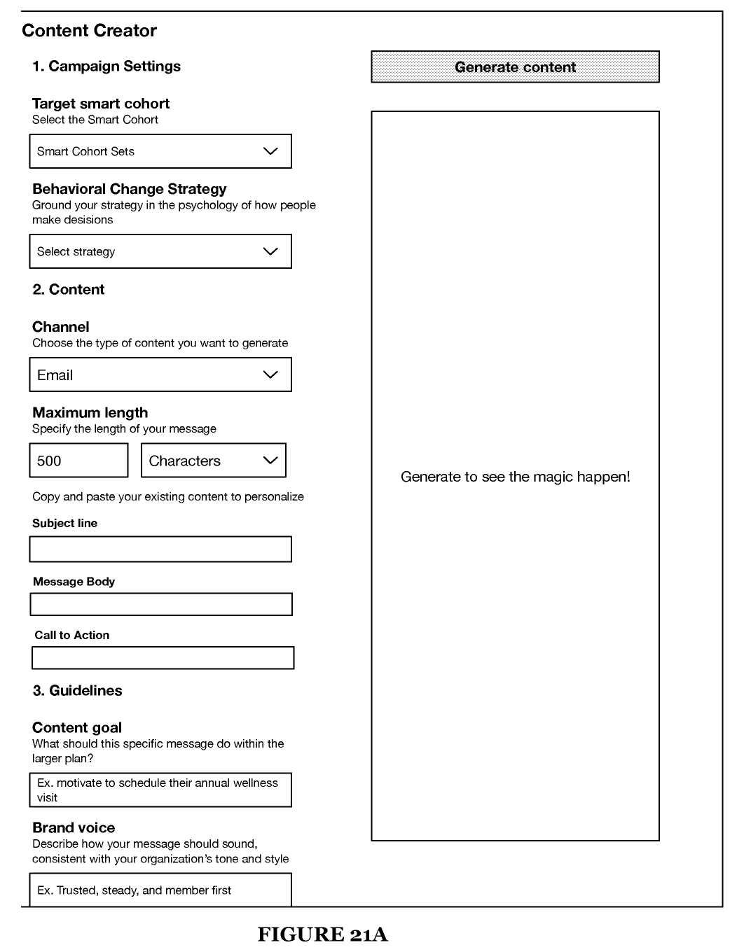

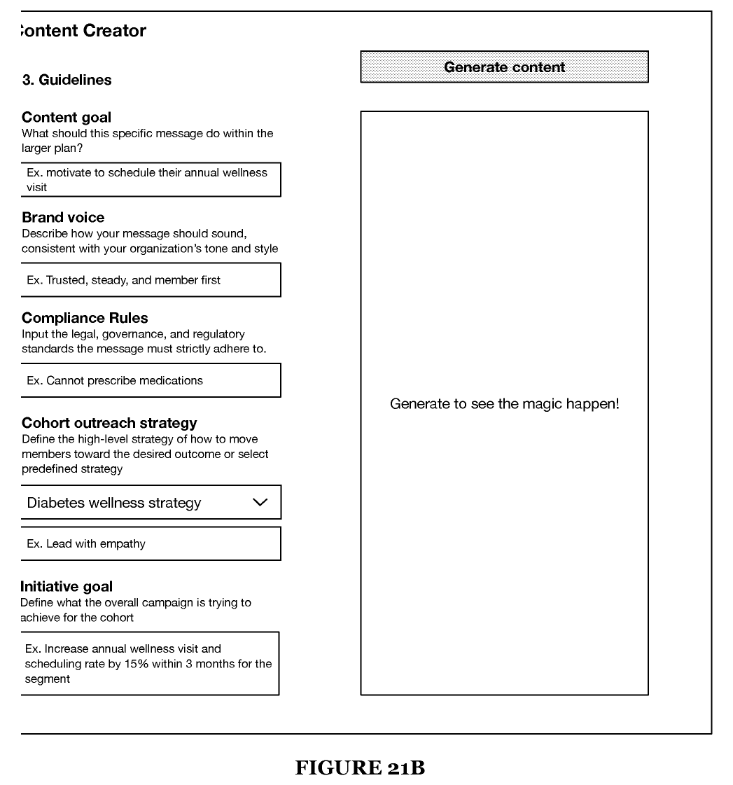

The user interface 700 functions to receive user inputs. The user interface 700 can receive one or more inputs from a user (e.g., media uploads, natural language, human readable configuration files, programs, executables, etc.), display data, display one or more outputs (e.g., model outputs, cohorts, features, feature values, analyses, etc.), display any other parameters, and/or otherwise function. Examples of the user interface are shown in FIG. 13A, FIG. 13B, FIG. 19, FIG. 20, FIG. 21A, and FIG. 21B. The user interface 700 can be customizable for a customer, or can include one universal format.

The user interface 700 can include a set of display elements. The set of display elements can include structural elements (e.g., panels, tabs, view, etc.), navigation elements (e.g., menu, toolbar, pagination control, etc.), input elements (e.g., button, toggle, dropdown, text field, search field, file uploader, slider, etc.), display elements (e.g., text block, image, chart, map, label, metric summary, etc.), selection elements (e.g., filter, sort, range, category selector, etc.), state elements (e.g., error, progress, etc.), and/or any other display elements.

The user interface 700 can receive an input at a display element that can trigger an action, stop an action, and/or otherwise perform any other action. In a variant, receiving data at a user upload can trigger the method (e.g., in S100). In a variant, the user interface can display a set of display elements (e.g., display elements, input elements, and/or selection element), and receiving data at the user interface (e.g., natural language user input) can cause a response (e.g., generating a strategy, generating content, etc.).

However, the user interface 700 may be otherwise configured.

The campaign executor functions to implement the outreach strategy. The campaign executor can include an orchestrator, agent (e.g., LLM agent), and/or other model. The campaign executor can alternatively be a manual executor. The campaign executor can call APIs and/or otherwise programmatically implement each step in the outreach strategy.

In an example, the campaign executor can call the API of a mass-mailer with the step's content and set of addresses for the members in the cohort when a trigger event is detected.

However, the campaign executor may be otherwise configured.

All or a subset of the models used in the system can use classical or traditional approaches, machine learning approaches, and/or other approaches. The models can include regression (e.g., linear regression, non-linear regression, logistic regression, etc.), decision tree, LSA, clustering, association rules, dimensionality reduction (e.g., PCA, t-SNE, LDA, etc.), neural networks (e.g., CNN, DNN, CAN, LSTM, RNN, encoders, decoders, deep learning models, transformers, generative models, diffusion models, etc.), ensemble methods, optimization methods, classification, rules, heuristics, equations (e.g., weighted equations, etc.), selection (e.g., from a library), regularization methods (e.g., ridge regression), Bayesian methods (e.g., Naiive Bayes, Markov), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, and/or any other suitable architecture. The models can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of attention layers (e.g., cross-attention layers, self-attention layers, etc.); and/or have any other suitable architecture. The models can include less than 10, tens, hundreds, thousands, tens of thousands, hundreds of thousands, and/or any other number of parameters (e.g., weights, biases, etc.). The models can extract data features (e.g., feature values, feature vectors, high-dimensional features, embeddings in a high-dimensional space with hundreds or thousands of dimensions, human-unintelligible features, etc.) from the input data, and determine the output based on the extracted features. However, the models can otherwise determine the output based on the input data.

The system models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels), negative training sets (e.g., a set of data with true negative labels), and/or any other suitable set of data.

Any model can optionally be validated, verified, reinforced, calibrated, or otherwise updated based on newly received, up-to-date measurements; past measurements recorded during the operating session; historic measurements recorded during past operating sessions; or be updated based on any other suitable data.

Any model can optionally be run or updated: once; at a predetermined frequency; every time the method is performed; every time an unanticipated measurement value is received; or at any other suitable frequency. Any model can optionally be run or updated: in response to determination of an actual result differing from an expected result; or at any other suitable frequency. Any model can optionally be run or updated concurrently with one or more other models, serially, at varying frequencies, or at any other suitable time.

However, the system can be otherwise configured.

### 4. Method

As shown in FIG. 1, variants of the method can include: determining datasets for each of a set of members S100; optionally processing the data S200; determining a set of features S300; determining a set of cohorts S400; optionally generating content strategy S420; and generating content for a cohort S500. The method functions to generate personalized content based on health-related information.

In a first variant, the method can function to stratify a member population into cohorts and generate personalized content for each cohort. In a second variant, the method can function to determine personalized content and optionally a personalized content delivery strategy for each member in a member population. However, the method can perform other functionalities.

In an example, the method can include: receiving (e.g., from a healthcare organization) data for a set of members (e.g., consumers, patients, etc.), optionally enriching the data, determining a set of features based on the data, determining a set of cohorts including members in the set of members, optionally generating a set of cohort metrics for each cohort in the set of cohorts based on aggregated member features of members in the cohort, and generating personalized content for each cohort based on the cohort information. In a specific example, enriching the data can include correlating member addresses to social determinants of health data and/or by inferring missing data. In a specific example, determining a set of features can include wherein a member feature in the set of features is associated with a member in the set of members. In a first specific example, cohorts can be generated from the set of members by ranking features (e.g., human-interpretable features) of the data based on their correlation to a target variable (e.g., a metric), and systematically assigning members to cohorts based on the ranked features (e.g., in a 'waterfall' approach). In a second specific example, cohorts can be generated from the set of members by tuning a model based on the data, and clustering the members in a latent space of the tuned model. In a specific example, generating content performed by a set of large language models according to a set of guardrails, wherein the content can be evaluated by a guard model after generation for adherence to the guardrails.

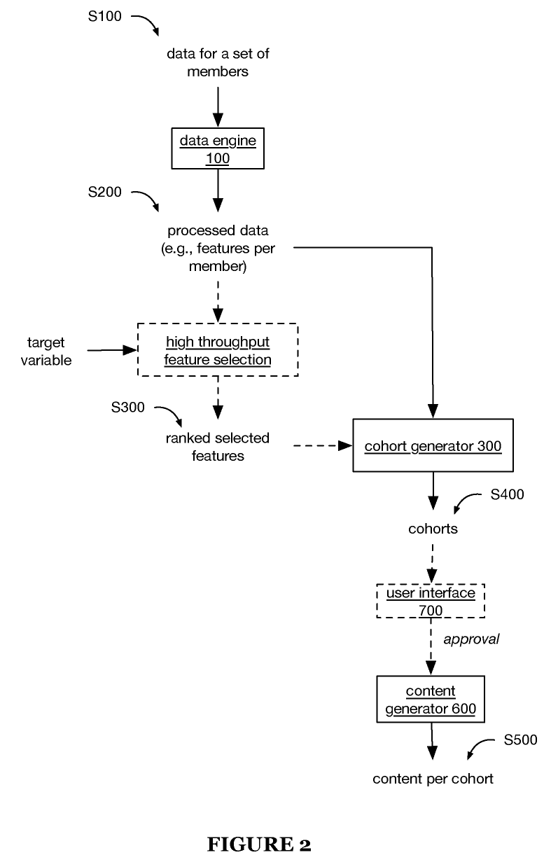

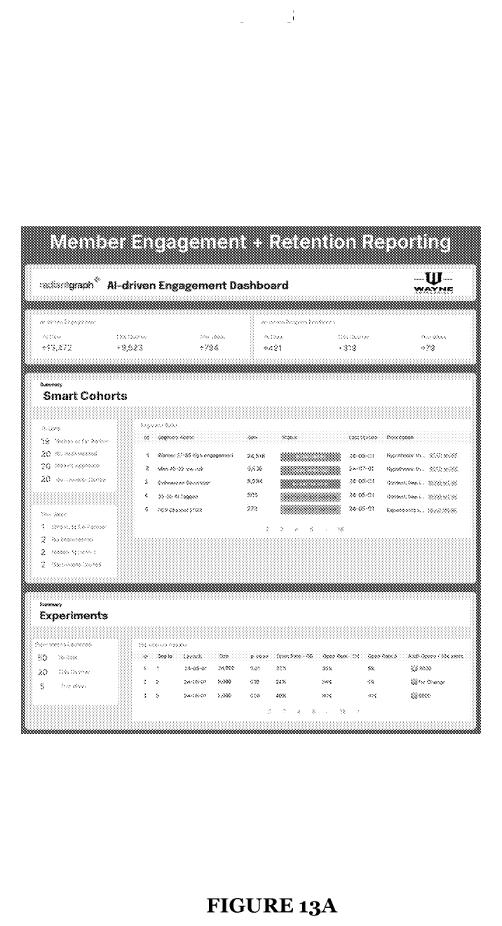

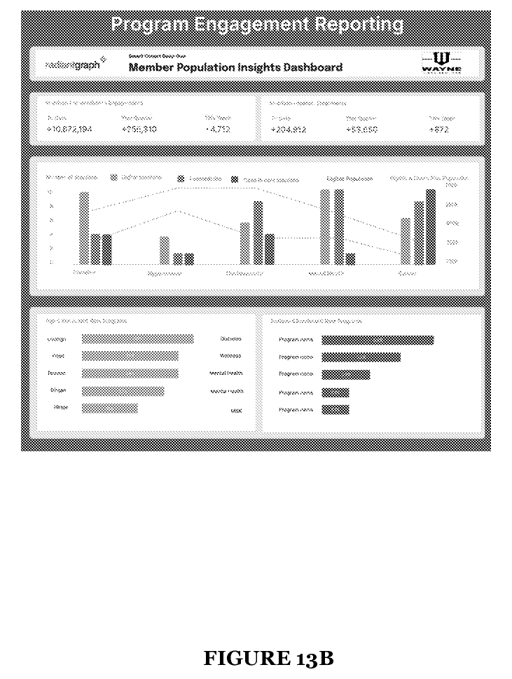

Examples of the method are shown in FIG. 1, FIG. 2, and FIG. 10B.

All or portions of the method can be performed in real time (e.g., responsive to a request), iteratively, concurrently, asynchronously, periodically, and/or at any other suitable time. All or portions of the method can be performed automatically, manually, semi-automatically, and/or otherwise performed.

All or portions of the method can optionally be performed for each of a set of users, for each of a set of members, for each of a set of target variables, and/or otherwise performed. In an example, users include healthcare organizations. Specific examples of healthcare organizations include: healthcare provider companies, health plan companies, member engagement companies, healthcare providers, hospitals, and/or any other healthcare organizations. Additionally or alternatively, users can include data engineers, data scientists, data analysts, prompt engineers, and/or any other user. All or portions of the method can be performed automatically, manually, semi-automatically, and/or otherwise performed.

All or portions of the method can optionally be performed using one or more target variables (e.g., target use cases). In an example, the target variable can be a metric (e.g., objective; e.g., whether the member has or will convert) used to evaluate a member and/or a member cohort. Specific examples of using the target variable include: selecting features that have an impact on the target variable, selecting a cohort (e.g., for content generation) based on the (known or predicted) percentage of members in the cohort that satisfy a criterion associated with the target variable, analyzing cohorts with respect to the (known or predicted) target variable value for the members in the cohort, generating content based on the target variable, and/or otherwise using the target variable and/or values thereof in all or portions of the method. The number of target variables used for an iteration of all or portions of the method (e.g., for determining cohorts, for generating content, etc.) can be between 1-10 or any range or value therebetween (e.g., 1, 2, greater than 2, etc.). The number of target variables can alternatively be greater than 10. The target variable can be qualitative, quantitative, relative, discrete, continuous, a classification, numeric, binary, and/or be otherwise characterized.

The target variables can include enrollment (e.g., a metric evaluating whether a member enrolls in a health plan), future enrollment (e.g., a metric evaluating whether a member will enroll in a health plan), conversion (e.g., a metric evaluating whether a member converts to one or more target health plans), future conversion (e.g., a metric evaluating whether a member will convert to one or more target health plans), engagement (e.g., a metric evaluating whether a member engages with emails), future engagement (e.g., a metric evaluating whether a member will engage with marketing), current diagnosis (e.g., a metric evaluating whether a member has a target diagnosis), future diagnosis (e.g., a metric evaluating whether a member will be diagnosed with a target diagnosis in the future), and/or any other target variables.

In a first specific example, a target variable can be a binary metric (e.g., previously enrolled or did not enroll in a health plan; predicted to enroll or predicted to not enroll in a health plan; previously engaged with emails or did not engage with emails; predicted to engage with emails or predicted to not engage with emails; previously diagnosed or not diagnosed with a target disease; predicted to be diagnosed or not diagnosed with a target disease; etc.), a probability metric (e.g., the likelihood that the member will enroll in the health plan; the likelihood that the member will engage with an email; the likelihood that the member will be diagnosed with the disease in the future; etc.), and/or any other metric.

A member can optionally be associated with a value for the target variable (e.g., 'enrolled'; 'not enrolled'; '90% likely to enroll'; 'positive breast cancer diagnosis'; 'negative breast cancer diagnosis'; '20% likely to develop breast cancer in the next 10 years'; etc.).

The target variable can be manually determined, predetermined, determined by a user (e.g., the customer provides the target variable), and/or otherwise determined.

The values for the target variable can be determined based on the member data, determined (e.g., predicted) using a predictive model, and/or otherwise determined.

The method can be performed using the system discussed above, and/or using any other suitable system. In a specific example, the system can be a platform that interfaces with one or more users (e.g., healthcare organizations), wherein the platform can receive inputs from the user(s) and/or provide outputs to the user(s).

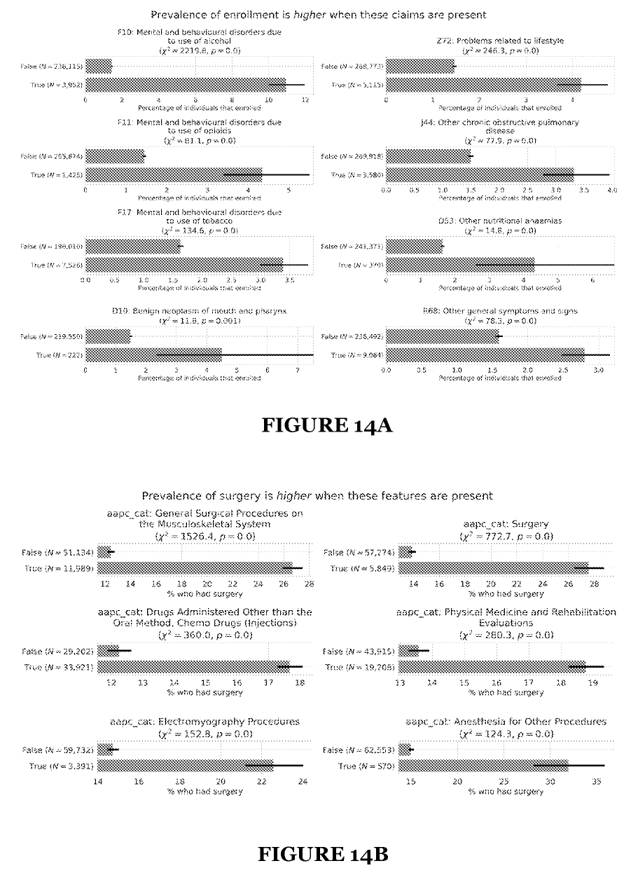

An example of the platform is shown in FIG. 11.

The specific examples of inputs received from a user can include a set of members, data for the set of members, a target variable and/or value thereof, content guidelines, and/or any other suitable inputs.

Specific examples of outputs provided to a user can include: processed data (e.g., enriched data), selected features, cohorts, information associated with a cohort (e.g., description, feature values, predicted target variable values, etc.), generated content, and/or any other suitable outputs.

Determining datasets for each of a set of members S100 functions to collect data on a set of members. The datasets for S100 can be determined from a user input, a database retrieval, a look up, and/or any other suitable method. S100 can be repeated until a stop condition is met, performed once, and/or otherwise performed. In an example, determining data for a set of members (e.g., healthcare members) can include receiving data from a user (e.g., healthcare organization) for each member of a set of members. In specific examples, the members can be potential members associated with the user, current members associated with the user, a combination thereof, and/or any other population. In a specific example, a member is a patient (e.g., with a diagnosis). In another specific example, a member does not have a diagnosis.

Examples of data (e.g., member data) can include claims data (e.g., claim history), health data (e.g., diagnoses, patient history, lab tests, biometric values, medication history, medical history, ICD data, outcome data, etc.), demographic data (e.g., location information, gender, age, race, income level, etc.), self-reported data (e.g., activity level), churn data, engagement data (e.g., clicking on links, opened emails, responded to emails, etc.), health plan data (e.g., current and/or previous health plans a member has engaged), data on interactions with a healthcare organization, eligibility data, subscriber status, product usage, personal preferences, social determinants of health (SDoH) data (e.g., as described in S200), metadata thereof, optional target variable data (e.g., engagement history, etc.), sentiment data, operational data (e.g., support tickets, grievances), and/or any other information. In a specific example, location information can include: zip code, partial or complete address, country, county, and/or any other member location information.

The data can optionally include one or more values (e.g., historical values) for the target variable(s). For example, the data can include data for current and/or past members, wherein the data includes current and/or historical values for the target variable(s). In an illustrative example, for an engagement target variable, the data can include historical engagement with email content. In another illustrative example, for a conversion target variable, the data can include historical conversion from a first health plan to a second health plan.

The data can be received from claims and eligibility records, clinical and institutional providers, marketing and engagement platforms, core administrative systems, self-reported data, AHRQ dataset, the census bureau, CDC, ATSDR, USDA economic research service, political data, and/or any other data source.

However, data can be otherwise determined in S100.

The method can optionally include processing the data S200, which functions to enrich, clean, detangle, aggregate, synthesize, and/or otherwise adjust the data for one or more downstream processes (e.g., for feature selection). S200 can be performed after S100 and/or at any other time. An example of S200 is shown in FIG. 10A.

Processing the data can include deduplicating, standardizing, filtering, normalizing, extracting features, transforming, aggregating, downsampling, fitting, smoothing, denoising, extracting statistical metrics, validating, and/or any other processing methods. S200 can be performed using the data engine, a set of rules, a look up table, a model, and/or any other suitable method.

In a first variant, the data can be processed using a data engine. The same data engine can be applied to all the population types (e.g., general populations, sub-populations, un-cohorted populations, cohorted populations, etc.) and/or users. Alternatively, different data engines can be applied to different population types and/or users (e.g., the data engine can be specific to a particular user and/or population type).

In a second variant, can include filtering data (e.g., temporally, semantically, etc.) to determine target events (e.g., most relevant events). In an example of the second variant, thresholds for filtering the data can be dynamic, configurable, and/or predetermined.

However, the enrichment can be otherwise performed

In a first variant, processing the data S200 can include enriching member data (e.g., collected via S100) using supplemental data. The supplemental data is preferably retrieved from a database (e.g., a third-party database), but can additionally or alternatively be received from a user, determined using a model, and/or otherwise determined. The specific examples of data sources used to determine the supplemental data can include an Agency for Healthcare Research and Quality (AHRQ) dataset, a census dataset, an election results dataset, a Rural-Urban Commuting Area Codes (RUCA) dataset, a PLACES dataset, a social vulnerability index (SVI) dataset, a private dataset, an external dataset (e.g., purchased from a vendor), and/or any other database and/or dataset therefrom. The supplemental data can include SDoH data (e.g., SDoH signals), claims data, any external data (e.g., separate from data received from the user via S100), descriptors, risk factors, and/or any other data. In examples, SDoH data include: AHRQ metrics, census metrics, national election results, RUCA metrics, income metrics (e.g., income level), access barriers, social vulnerability index, weather data, socioeconomic status, education level, employment status, health risk factors (e.g., for conditions such as diabetes, heart disease, etc.), propensity scores, morbidity rates associated with geographic data, health behaviors (e.g., physical activity, diet, etc.), and/or any other suitable data.

In an example, enriching the supplemental data can include associating ICD codes with the respective code descriptions (e.g., in natural language). In specific examples, one or more features can be extracted from the ICD code itself, the respective code descriptions, and/or other information.

In a second variant, the data (collected via S100) can be used to determine supplemental data associated with the member. For example, location data, demographic data, and/or any other member data can be correlated with supplemental data. The addition of supplemental data can be associated with and/or triggered by a data field of a member, a data value for a member, a metric derived from a data field and/or value of a member, and/or any other association or trigger. The data field of a member can include claims data, a diagnosis field, and/or any other data field. The data value for a member can include a diagnosis, a demographic, family history, an address, an ICD diagnosis code, and/or any other data value. The metric derived from a data field and/or value of a member can include a number of appointments scheduled, a number of screenings scheduled, and/or any other metric.

In a specific example, when the member data (collected via S100) includes location information (e.g., zip code, address, etc.) for each member, the member data for a given member can be paired with supplemental data associated with the location information (e.g., average income associated with the zip code, past medical claims associated with the address and/or name, etc.).

In an example, supplemental data can include descriptors and ICD diagnosis codes can be enriched with descriptions, groupings, domains, and/or hierarchical categories. In a specific example, ICD-9 and/or ICD-10 codes can be mapped to categories. In a specific example of supplemental data including risk factors, claims data can be enriched with comorbidity patterns and risk predictions.

In a third variant, when the member data is missing data (e.g., gender labels for a member, target variable values, etc.) or conflicting data (e.g., variations in names, addresses, and other identifiers), the missing and/or conflicting data can be inferred. The missing and/or conflicting data can be inferred based on the supplemental data, a set of data associated with the member, a set of data input by the user, temporally adjacent values for the data field, and/or any other suitable data source. In a first specific example, the missing data can be predicted using statistical methods, using a predictive model (e.g., a propensity model), and/or otherwise determined. In a second specific example, null versus false values can be inferred based on context. When none of the members in the set have a particular characteristic (e.g., a diagnosis code), it can be treated as null (e.g., unknown, no data available) and be excluded from calculations. When a subset of members have the characteristic (e.g., a diagnosis code) but the rest of the set does not, the missing characteristic can be treated as false (e.g., no condition). In a third specific example, conflicting data across different data sources (e.g., claim files, eligibility files, etc.) can be resolved using probabilistic matching methods. Conflicting data can include different spellings of names, historical addresses, missing middle initials, character errors in data entry, and/or any other record associated with a member. In a fourth specific example, members can be matched to associated data from different data sources, (e.g., insurance claims, electronic health records (HER), and/or pharmacy records) when traditional identifiers may be missing or inconsistent. The specific example can include generating deterministic unique identifiers for matched records.

As used herein, "data" can refer to processed data (e.g., enriched data) and/or unprocessed data.

However, S200 can otherwise process the data.

Determining a set of features S300 functions to identify a set of features associated with the target variable(s) for a member in the set of members. The features can preferably be based on a set of data associated with a member in the set of members, but can alternatively be based on a processed set of data associated with a member in the set of members, a set of data associated with a subset of members, or a set of data associated with a set of data associated with the set of members. The determined set of features can include: member feature sets (e.g., feature set per member); population feature sets (e.g., features for the population, member feature sets aggregated across the population's members; etc.); and/or any other set of features. The features can refer to a feature definition, a feature value, or a representation derived therefrom, unless otherwise specified.

S300 can be performed after S100, after S200, and/or at any other time.

Feature values can be qualitative, quantitative, relative, discrete, continuous, a classification, numeric, binary, natural language, and/or be otherwise characterized. Features are preferably semantic features (e.g., human-interpretable features), but can additionally or alternatively include non-semantic features. Features can be handcrafted, freeform, learned by a model, selected by model, and/or otherwise characterized.

In a first example, values for all possible features are extracted for a member.

In a second example, values for features that the member has values for are extracted.

In a third example, values for a predetermined subset of all possible features are extracted for the member. The predetermined subset of all possible features can be: features that historically have a high correlation, lift, or propensity with a predetermined target outcome (e.g., engagement with a product, conversion, etc.) for the campaign; user-selected features; and/or other features.

The features can be user-specific, target-specific, and/or can include a predetermined set of feature types. The feature values can be based on any data determined in S100 and/or data processed in S200, including: claims data, eligibility files, geographic data, engagement data, demographics, and/or any other data. In an example, the feature values can include extracting a feature based on a description associated with an ICD code.

The values for the features can be elements of the data (e.g., the data includes feature values for each member), data fields of the data, extracted from the data, predicted based on the data (e.g., using a predictive model), and/or otherwise determined based on the data.

In illustrative examples, features can include whether the member has filed a specific claim, age, gender, estimated income, geographic location, access barriers (e.g., determined based on geographic location), subscriber status, eligibility for a health plan, conversion likelihood (e.g., determined using a predictive model), classifications thereof, medical history, database field names, and/or any other suitable member data features. The medical history can include International Classification of Diseases (ICD) code, Current Procedural Terminology (CPT), laboratory test history (e.g., diagnostics, screenings, etc.), and/or any other medical history. In a specific example, the ICD code can include a hierarchy of characters out of an ICD code hierarchy.

In variants, determining a set of features S300 optionally includes selecting a subset of the feature set; and optionally predicting a value for the target variable based on a set of features.

Selecting a subset of the feature set, which functions to identify the best (e.g., highest correlation, highest lift, etc.) features to use for creating cohorts. Subsetting the feature set can additionally be used to determine which features to extract content-generation information (e.g., cohort demographic distribution, etc.) from. Subsetting the feature set can additionally reduce the context length of the model inputs (e.g., for cohort generation, content generation, etc.). Subsetting the feature set can additionally improve the attention of the model (e.g., to focus the model on the more important features). Subsetting the feature set is preferably performed by the feature selector, alternatively by the data engine and/or any other system.

The feature set subset can be treated as the cohort creation feature set (e.g., the set of features used to create one or more cohorts), or be otherwise treated. The cohort creation feature set is preferably a subset of the population feature set, but can be otherwise defined. The selected features can be a subset of features associated with (e.g., correlated to, indicative of, etc.) the one or more target variables (e.g., be specific to the target variable). The features can be selected based on the target variable(s), the data (e.g., processed or unprocessed data), based on a user input (e.g., a user specifies features to select, such as gender, age, etc.), a combination thereof, and/or based on any other suitable information.

In a first variant, selecting a subset of the feature set S300 can include analyzing the features and selecting features based on the analysis (e.g., a high throughput feature selection). For example, the data can be analyzed using one or more statistical tests (e.g., in parallel), and one or more features correlated with one or more target variables can be selected. In this variant, selecting the feature subset can include selecting a subset of an initial group of determined features based on an analysis.

The features can be selected based on characteristics including: widest coverage of the set of members; highest number of segments (e.g., subsets) of the set of users ; lowest number of segments of the set of users; number of users associated with a feature; predictive power (e.g., positive correlation, negative correlation, strength of correlation); statistical significance (e.g., chi-square tests, P-value thresholds, Bonferroni correction for multiple comparisons, etc.); model performance (e.g., whether including the feature improves overall model accuracy); business interpretability (e.g., actionable for customization, explainable to users, compliant with privacy, etc.); data quality; dimensionality reduction; and/or otherwise selected.

The features can be determined and/or constrained by a set of guardrails. An illustrative example of a guardrail can include not using combinations of identifiable data or not including medical prescriptions.

In a first example, multiple chi-squared tests can be performed (e.g., in parallel) to analyze associations between pairs of features (e.g., a chi-squared test can be performed for each feature–feature pair).

In a second example, multiple chi-squared tests can be performed (e.g., in parallel) to analyze associations between a feature and a target variable (e.g., a chi-squared test can be performed for each feature–target variable pair). In a specific example, features with greater than a threshold correlation (e.g., positive correlation or negative correlation) to a target variable can be selected. In a second example, statistically significant features can be retained while others are discarded.

In a third example, subsetting the feature set can include: determining a target variable (e.g., conversion, enrollment, churn, etc.); identifying members with historical engagement with the target variable; retrieving the feature sets for each of the identified members (e.g., set of non-null features; set of valued features; etc.); optionally convert the historical engagement for the target variable (e.g., churn / not churn) and the feature values for the retrieved feature sets into binary variables; and determining an association metric (e.g., correlation, lift, mutual information, etc.) between the target variable and the features (and/or feature values). The members are preferably from the same population, but can alternatively be from a different population. In a first specific example, the system can determine that the BMI feature is strongly positively correlated with enrollment into a diabetes program, height is uncorrelated, and daily physical exercise is strongly negatively correlated with enrollment into the diabetes program. In a second specific example, the system can determine that a BMI > 35 has a strongly positively correlated with enrollment into a diabetes program, BMI between 25-35 is uncorrelated, and BMI <25 is strongly negatively correlated with enrollment into the program.

In a fourth example, the subset of features is retrieved based on the target variable and/or target outcome.

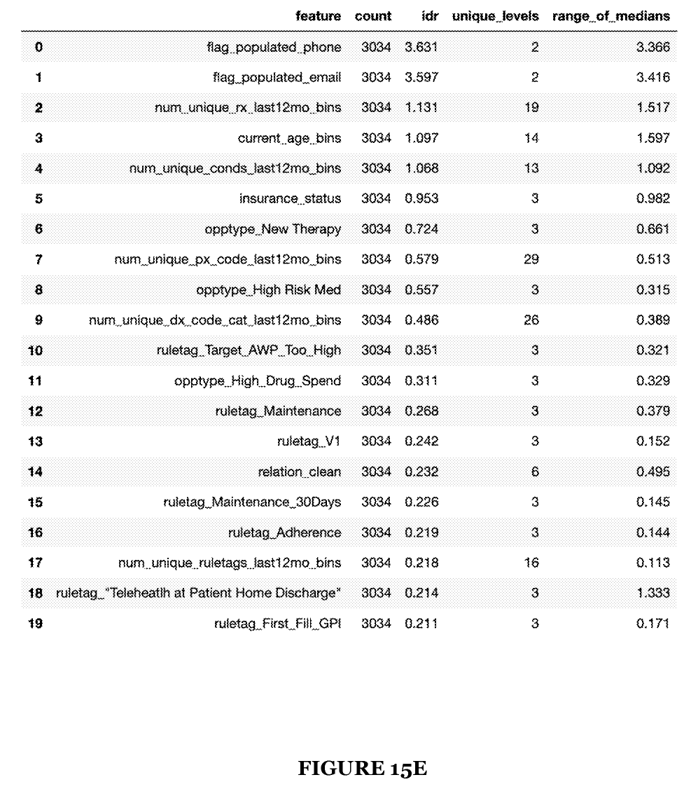

Examples of determining a set of features are shown in FIG. 14A and FIG. 14B.

In a second variant, selecting a subset of the feature set can include training a predictive model to predict a target variable value based on feature values, and selecting features based on the trained model. For example, each feature can correspond to a weight in the predictive model, wherein the weights are adjusted during training; the features with (learned) weights above a threshold can optionally be selected.

The features can optionally be ranked according to their association metric (e.g., a correlation metric, a p-value, lift, etc.) to the one or more target variables. In variants, S300 can optionally include filtering for the strongest correlations (e.g., positive or negative) and/or filtering out uncorrelated, weakly correlated, or independent features to subselect the feature set (e.g., create a feature subset for cohort generation, etc.).

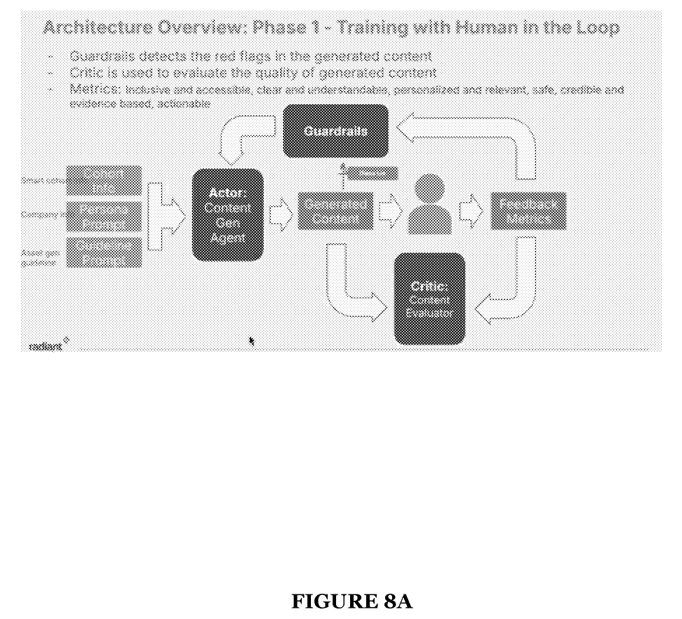

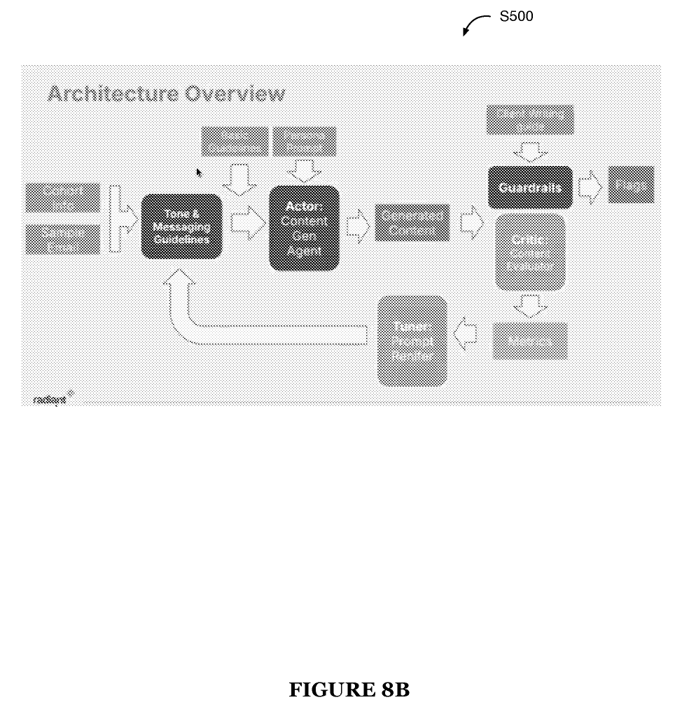

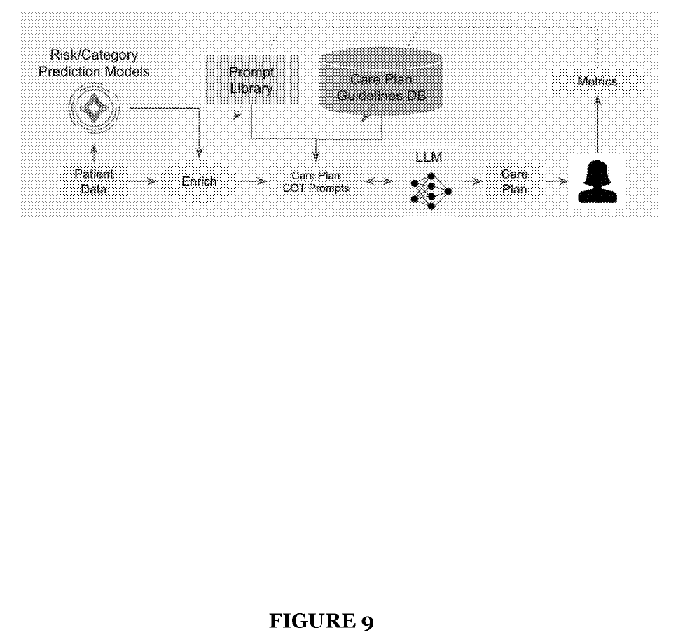

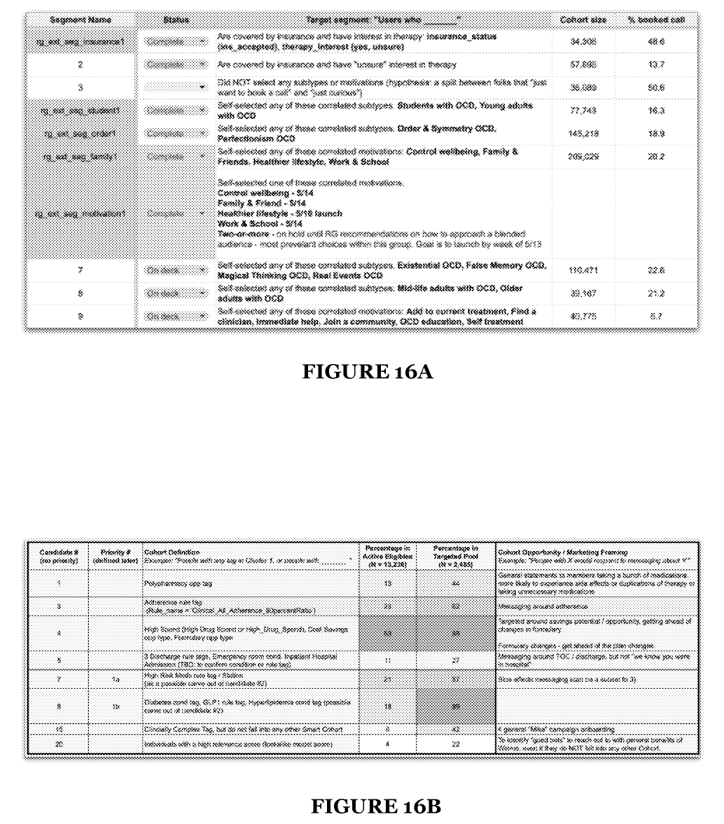

Examples are shown in FIG. 7A and FIGS. 15A-15E.

However, features can be otherwise determined.

S300 can optionally include predicting a propensity value for the target variable based on a set of features. The propensity value can be predicted for each member in the set of members, for a cohort, and/or for any set of members. The propensity value is preferably determined by the propensity model, but can additionally and/or alternatively be performed using a set of rules, or any other model. An example is shown in FIG. 4D. The predicted target variable value can include: an expected engagement value per delivery mechanism, a probability score for engagement, and/or any other value. In an example, the predicted value can describe whether a member is anticipated to engage with a piece of content or an outreach channel (e.g., a call). The predicted value can be used to select which outreach strategy to use for a given member, select which candidate content to use for a given member, and/or otherwise used.

The predicted value can be determined for a target variable or goal (e.g., screening participation), a response to a given outreach modality (e.g., call, email, text, etc.), and/or any other strategic variable. The target variable, goal, outreach modality, and/or strategic variable value can be selected by a user, automatically determined (e.g., from a campaign strategy, etc.), and/or otherwise determined.

The target variable value can be predicted based on cohort metrics, member features, cohort features, user inputs (e.g., guardrails, targets, goals, etc.), a strategy, brand strategy, resource constraints, and/or any other suitable basis. In a specific example, predicting a value for a target variable can include predicting a propensity score for a member in a cohort. In another specific example, predicting a value for a target variable can include predicting a propensity score for a member in a cohort and associating the propensity score with the cohort. In a specific example, predicting a value for a target variable can include predicting a propensity score for a cohort (e.g., based on a set of cohort metrics). In a specific example, predicting a variable based on feature values can include: determining an engagement probability score for each member in a set of members; determining a subset of users based on comparing probability scores to a predetermined threshold; and/or otherwise determined.

However, a set of propensity scores can be otherwise determined.

However, determining a set of features S300 may be otherwise performed.

Determining a set of cohorts S400 functions to identify members that can be grouped for content generation associated with the target variable(s). Additionally or alternatively, determining a set of cohorts can function to: identify members for content generation (e.g., where only a subset of members are provided content) and/or for any other use case.

The number of members within a cohort can be between 1-100,000 or any range or value therebetween (e.g., at least 2; at least 10; at least 100; at least 10,000; etc.). The number of members within a cohort can alternatively be greater than 100,000. The number of members within a cohort can be determined during S400, predetermined (e.g., the number of members within a cohort can be set within a predetermined range), and/or otherwise determined. The number of cohorts can be 1–1,000 or any range or value therebetween (e.g., at least 2; at least 5; at least 10; less than 100; less than 50; etc.), but can alternatively be greater than 1,000. The number of cohorts can be determined during S400, predetermined (e.g., the number of cohorts can be set prior to determining the cohorts), and/or otherwise determined. Preferably, each member in the set of members is assigned to a single cohort (e.g., cohorts include nonoverlapping groups of members), but can alternatively be assigned to multiple cohorts or no cohorts.

A plurality of cohorts are preferably generated for the same target variable (e.g., for different values of the target variable); alternatively, a single cohort can be generated for a given target variable. Different cohorts in the plurality of cohorts (e.g., for a target variable) are preferably mutually exclusive (e.g., the cohorts do not overlap; members are not concurrently in two cohorts at the same time), but can alternatively overlap or be mutually distinct (e.g., cohorts are mutually different but can overlap).

In a first example, each cohort can share a set of features (e.g., field name), alternatively, cohorts can have non-overlapping sets of features. In a specific example, cohorts can be determined based on combinations of the feature values for a feature set including: age range, gender, diabetes status, and family diabetes history.

In a second example, each cohort can share a set of feature values, alternatively, cohorts can have non-overlapping sets of features values. In a specific example, a first cohort contains people with diabetes, and a second cohort contains people without diabetes with no overlap with the first cohort.

The plurality of cohorts preferably collectively encompass more than a threshold proportion of the overall member population (e.g., more than 50%, 60%, 70%, 80%, 90%, etc.), but can alternatively encompass less.

The different cohorts can be generated based on the same feature sets (e.g., be distinguished based on different feature value permutations), different feature sets (e.g., be distinguished by using different feature permutations), and/or be otherwise differentiated.

A member can concurrently be in multiple cohorts (e.g., for different target variables), or only be in a single cohort.

Each cohort preferably includes multiple members, but can alternatively include a single member. In a first example, the same content and outreach strategy (e.g., series of outreach channels) is determined for all members of a given cohort. In a second example, the same content but different outreach strategy is determined for different member subsets of a given cohort (e.g., based on each member's propensity for a given outreach channel). In a third example, different content and outreach strategies are determined for each individual member (e.g., wherein each of the plurality of cohorts includes a single or low number of members).

S400 can be iteratively performed, using different sets of features and/or values thereof in each iteration to generate different sets of cohorts; performed once; and/or performed any other number of times. The final set of cohorts (e.g., used in S500) can be selected based on: the number of members in each cohort, the number of cohorts, a statistical analysis performed on the cohorts (e.g., example shown in FIG. 7C), manually selected, and/or otherwise selected.

Members are preferably grouped into cohorts based on their respective feature values, but can alternatively be otherwise grouped into cohorts. In an example, the cohorts can be determined based on predicted target variables (e.g., expected value of conversion, propensity score, etc.) determined in S300.

The cohorts are preferably formed based on correlation with the target variable, but can additionally or alternatively be formed randomly, based on member-member similarity, based on correlation with an outreach strategy (e.g., a given outreach channel, a given series of outreach channels, etc.), based on a correlation with a specific piece of content, and/or otherwise determined. Cohorts can be formed based on correlation between member features to a cohort creation feature set.

Explainability and/or interpretability methods can optionally be used to determine relevant features and/or values thereof for each cohort.

Each cohort can optionally be associated with a description (e.g., human-interpretable descriptors, natural language descriptors, etc.). The description can be used in content generation, provided to a user (e.g., healthcare organization) for approval, and/or otherwise used. The description is preferably determined based on the set of features and/or values thereof corresponding to the cohort, but can alternatively be otherwise determined. For example, the description can be or include the feature values corresponding to the cohort.

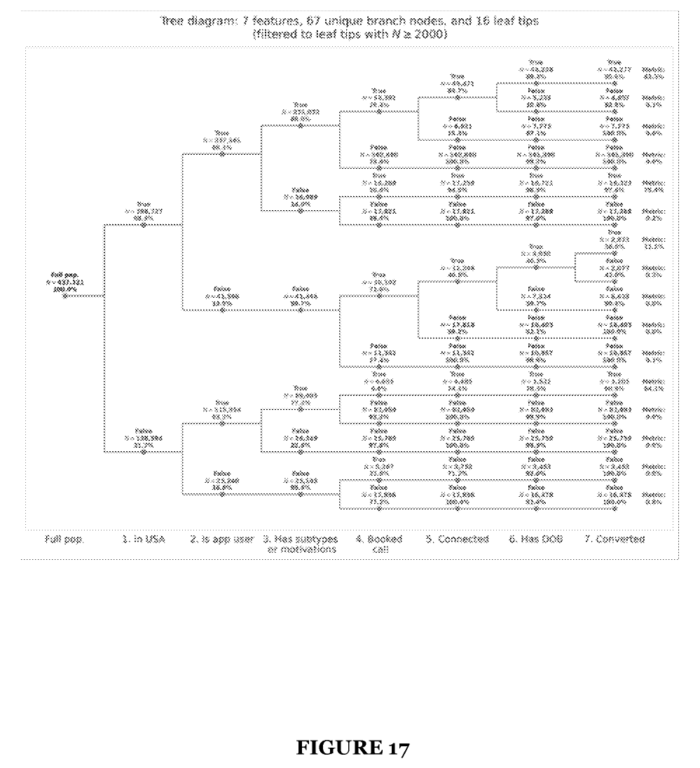

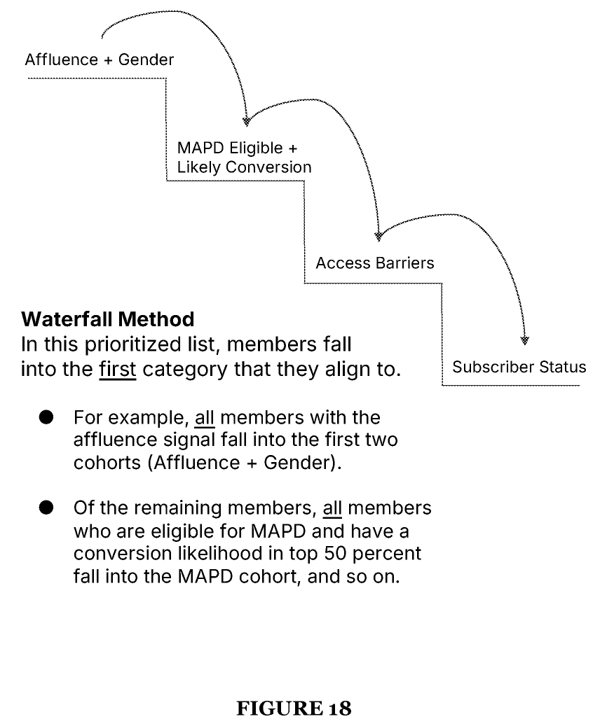

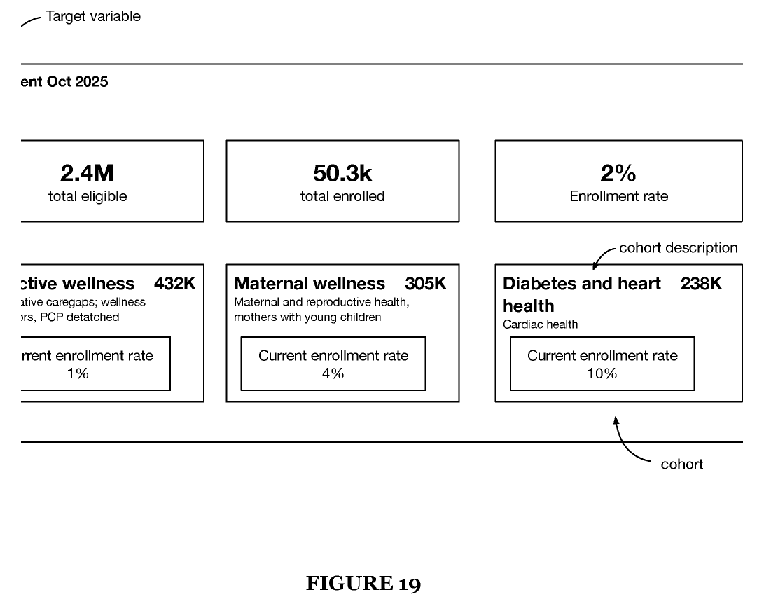

In a first variant, determining the cohorts includes grouping the set of members into cohorts based on ranked features (e.g., the ranked selected features from S300). In a specific example of the first variant, the cohorts can be generated in a series of stages via a 'waterfall approach' (e.g., where waterfall logic assigns each member to the first cohort that matches their feature values). Examples are shown in FIG. 3, FIG. 7B, FIG. 16A-16B, FIG. 17, and FIG. 18.

For example, a first cohort can be determined by selecting a first set of feature values for a first set of the ranked features (e.g., for all or a subset of the ranked features), wherein all members that satisfy the first set of feature values are assigned the first cohort; a second cohort can then be determined by selecting a second set of feature values (e.g., overlapping or nonoverlapping with the first set of feature values) for a second set of the ranked features (e.g., the same or different as the first set of ranked features; overlapping or nonoverlapping with the first set of ranked features), wherein remaining members (not assigned to the first cohort) that satisfy the second set of feature values are assigned the second cohort.

Each cohort can be selected to maximize the coverage of the remaining (e.g., unassigned, un-cohorted) member population, or be otherwise selected. In an example, the method can iteratively select features based on their ability to cover previously uncovered population segments, such as by applying a coverage mask to track which population members are already covered by selected features.

This process can optionally proceed for additional cohorts, progressively assigning subsets of remaining members to a cohort. The final cohort can optionally be a remainder cohort (e.g., with no corresponding feature values, encompassing all remaining members that have not been assigned a cohort).

Multiple cohorts can optionally be collapsed into a single cohort (e.g., based on the number of members in the respective cohorts). For each cohort, the set of features (e.g., a subset of the ranked features) and values thereof that correspond to the cohort can be: determined manually, determined based on the feature ranking, determined based on whether the feature has a positive or negative correlation to the target variable(s), determined based on previous cohort's features and/or values thereof, and/or otherwise determined.

The sets of features can optionally be determined based on feature ranking (e.g., where higher-ranking features are added to set(s) of features corresponding to earlier cohorts compared to the lower-ranking features). In a specific example of the first variant, the first set of features (for the first cohort) includes the highest-ranking feature, and the first set of feature values includes the feature value for the highest-ranking feature that has a positive correlation to the target variable(s). In this specific example, the second highest-ranking feature can be included in the first set of features and/or included in a later set of features.

In a first illustrative example, when the highest-ranking feature is affluence, where high affluence is positively correlated with a target variable, the first cohort can include members that are classified as 'high affluence. In this illustrative example, when the second highest-ranking feature is access barriers, where having access barriers is positively correlated with the target variable, the second cohort includes remaining members (e.g., not classified as high affluence) that are classified as having access barriers.

In a second illustrative example, when the highest-ranking feature is affluence, where high affluence is positively correlated with a target variable, and the second highest-ranking feature is access barriers, where having access barriers is positively correlated with the target variable, the first cohort can include members that are classified as 'high affluence' and 'having access barriers', and the second cohort can include remaining members that are classified as 'high affluence' and 'no access barriers.'

In a third illustrative example, when the highest-ranking feature is affluence, where high affluence is positively correlated with a target variable, and gender is manually selected to be an additional feature used, the first cohort can include members that are classified as 'high affluence' and female, and the second cohort can include members that are classified as 'high affluence' and male.

In a second variant, determining the cohorts can include generating a plurality of candidate cohort definitions (e.g., from the ranked selected features), then selecting a subset of cohort definitions from the plurality for final use. The subset of cohort definitions can be selected based on: collective coverage of the member population; disjointness; ranking of the features used in the respective cohort definition; and/or otherwise determined.

In a third variant, determining cohorts can include training (e.g., tuning) one or more models on the data (e.g., the processed data) and the one or more target variables, and clustering members in a latent space of the trained model (e.g., where each cluster is a cohort). In an example, multiple models are tuned on the data (e.g., propensity models, recommendation models, risk models, multi-embedded models, contextual models, etc.), wherein a model is selected from the multiple models for cohort generation (e.g., the most accurate model for the target variable is selected). A learned latent space in the selected tuned model can be used for clustering the members (e.g., where each member is embedded in the latent space). Additionally or alternatively, the cohorts can be determined by embedding the feature values (e.g., for all features, for the subset of features, etc.) into a latent space (e.g., non-human-readable latent space); and determining member clusters.

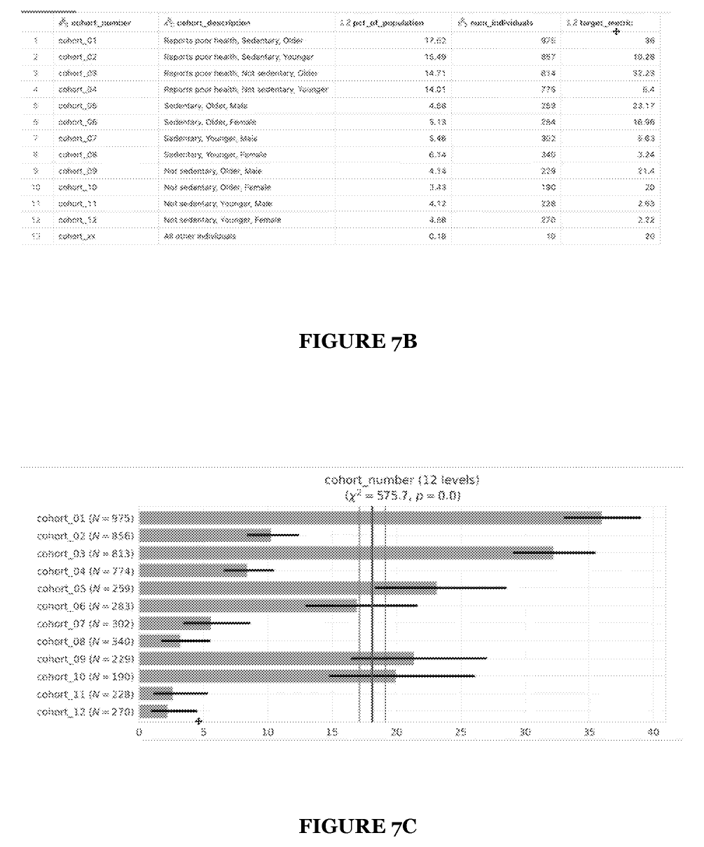

Examples are shown in FIGS. 4A-C and FIG. 6.

In an illustrative example, generating cohorts can include: preprocessing all possible permutations of all feature combinations; iteratively ranking the permutations based on a predetermined metric; and/or determining a set of best possible aggregates using a decision tree.

S400 can optionally include determining a set of cohort features for each cohort (e.g., as shown in FIG. 3). The set of cohort features can include: cohort metrics, descriptions, feature summaries, and/or other cohort features. In an example, the set of features associated with the cohort can be different from the set of features used to determine the cohort. The cohort description can include cohort metrics, qualitative descriptions, and/or any other descriptions.

The set of cohort metrics can include statistical measures of the cohort, geographic distribution of members in the cohort, SDoH data, and/or any other cohort metrics. The statistical measures of the cohort can include size of the cohort, median age of the cohort, probability of a member in the cohort having a morbidity, cohort alignment score, coverage of the set of member, engagement rate with a communication type, age range of cohort, distribution of diseases, and/or any other statistical measures.

In a variant, the set of cohort metrics can include metrics derived from features outside of a combination of population features used to define the respective cohort (e.g., outside of the cohort creation feature set, outside of the specific cohort creation features used to define the respective cohort, etc.).

The set of cohort features can be based on the members in the cohort, the features used to define the cohort, subsets of members in the cohort, the size of the cohort, and/or any other suitable basis.

However, the set of cohorts can be otherwise determined.

However, determining a set of cohorts S400 may be otherwise performed.

The method can optionally include generating content strategy S420, which functions to determine when and how content will be presented to a member (e.g., determine a "journey"). The content strategy (e.g., content delivery strategy, outreach strategy, campaign strategy, etc.) can be specific to a member, specific to a cohort (e.g., cohort strategy), specific to a subset of members in a cohort, and/or any other targeting approach. The content strategy can be generated by the strategy generator 500, alternatively can be generated by a human, a set of rules, and/or any other suitable method. The content strategy can be generated based on cohort features, cohort metrics, member features, enriched data about the set of members, predicting target variables (e.g., as determined in S300), and/or any other basis. The content strategy can be generated based on social determinants of health, from cohort creation feature values, from non-cohort creation feature values, and/or from any other information. An example of S420 is shown in FIG. 3.

In a first variant, generating content strategy S420 can include passing the set of cohort features (e.g., distribution of propensity scores, sub-clusters of propensity scores, distribution of member features, cohort description, etc.) and optionally a set of user preferences (e.g., brand strategy, content strategy, etc.) into a strategy generator (e.g., as a prompt), and receiving a predicted series of outreach steps, each with a predicted configuration. The predicted configuration can include: trigger dependencies (e.g., triggering condition); outreach channel for the step; optional content (e.g., final content, sample content); optional content guidelines (e.g., behavior change strategy, etc.); schedule; subset of members that the step applies to; and/or other configuration parameter.

In a second variant, S420 can include receiving the outreach strategy from the user (e.g., at a user interface).

In a third variant, S420 can include selecting an outreach strategy from a set of predefined outreach strategies, based on the set of cohort features, the set of user preferences, and/or other information.

However, generating content strategy S420 may be otherwise performed.

Generating content for a cohort S500 functions to generate content personalized to the cohort. S500 can optionally be iteratively performed for one or more cohorts (e.g., where different content is generated for different cohorts).

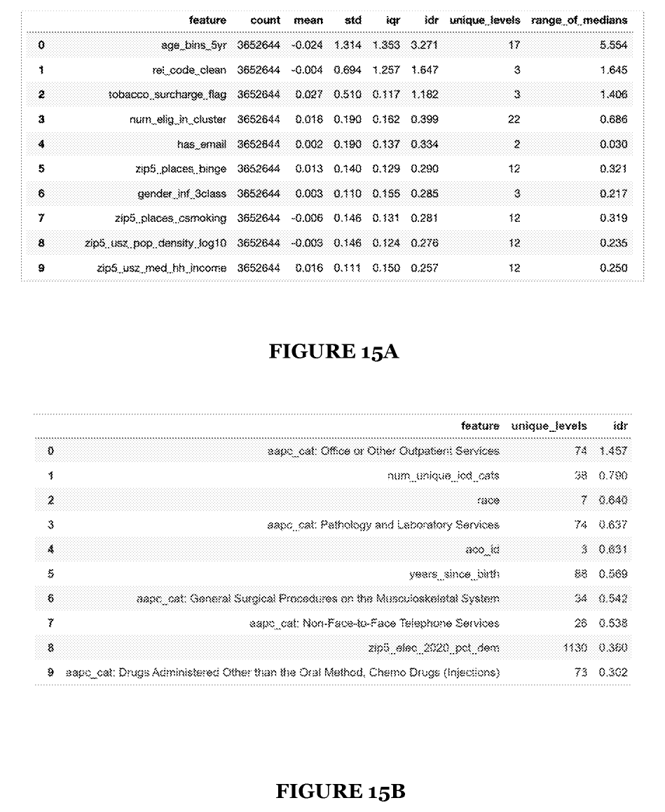

The content can include marketing materials, lifestyle recommendations, therapy recommendations, program and/or benefit recommendations (e.g., specific program offerings and/or benefits that might be of particular relevance and/or interest; for example a diabetes management program recommendation), communications (e.g., emails, voice call script, video, mailers, etc.), and/or any other healthcare content for the members in the cohort. An example is shown in FIG. 12.

In variants, S500 can use all or portions of the method disclosed in US Application No. 19/071,510 filed 03/05/2025, which is incorporated herein in its entirety by this reference. However, the content can alternatively be otherwise generated.

The content assets can be generated by the content generator 600, but can additionally or alternatively be generated by any other model or entity.

In a first variant, generating content can include aggregating content assets that have been pregenerated (e.g., by a human, by a model, etc.) and prevalidated (e.g., by a human, by a model, etc.). In examples, S500 can exclude generating new content. In other examples, S500 can generate new content.

In a first illustrative example, S500 can include: generating a strategy for a cohort; generating a video script; receiving validation from a human review; and based on the strategy and the video script, generating a video. In the example, the video can include an AI-generated human reading the script using the behavioral strategy, communication style, and content included in the strategy.

In a second illustrative example, S500 can include: generating a strategy for a cohort; generating a call script using content assets validated by human reviewers; calling a member; and reciting the script.

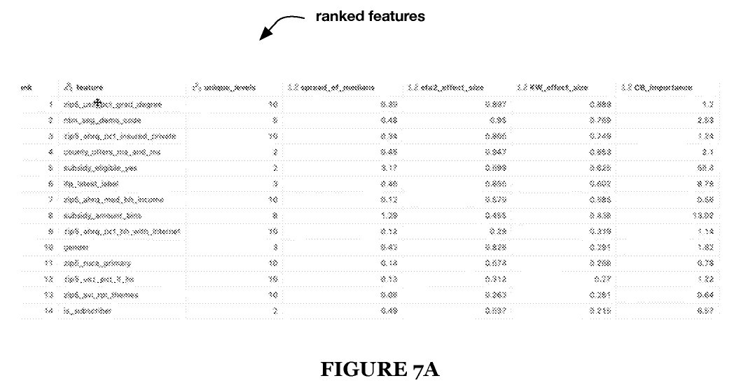

The content can be generated based on the set of cohort features (e.g., statistical metrics for non-cohort-defining features; statistical metrics for cohort defining features; statistical metrics for the cohort overall; etc.); the cohort description (e.g., the features and/or values thereof corresponding to the cohort, natural language summary, etc.), the one or more target variables; a writing guide (e.g., provided by the user); sample content (e.g., a sample email); user input (e.g., corrections, prompts, preferences, etc.); a chain of thought (CoT) prompt; a set of guardrails; brand strategy; content strategy; and/or any other information. Examples of inputs to the content generator are shown in FIG. 5 and FIG. 6.

In a variant, the set of cohort features can include features (and/or values thereof) not used for the selection of members for the cohort (e.g., features associated with the aggregate cohort rather than cohort creation features; non-cohort creation features). In a first example, if the cohort correlates to a feature value (e.g., a threshold percentage of members is classified with a shared psychographic group), that feature value can optionally be used for content generation. In a second example, the statistical measures (e.g., distribution, mean, median, standard deviations, etc.) of the non-cohort-defining features are used to generate the content for the cohort. In a third example, statistical measures for all features (e.g., all extracted features, all selected features, etc.) are used to generate content for the cohort. In a specific example, the content can be generated based on a prediction of what type of communication and/or content the cohort is expected to engage with the most.

The content generation can receive user input at any point in the generation process. In an example, content generation can be paused in response to a human intervention, wherein generation is restarted using the human input. The user input can include editing a script, modifying a prompt, adding new data, regenerating an output, modifying a piece of media, and/or any other user input.

Content can be generated based on a content strategy. The content strategy can include a trajectory of content, a content delivery mechanism, a schedule, a behavioral strategy, a communication style, and/or any other content strategy. The behavioral strategy can include a Behavior Change Wheel (BCW) framework, nudge theory, mindfulness-based approaches, risk communication, skill-building approaches, motivation-focused models, habit formation models, incentive approaches, and/or any other behavioral strategy. The communication style can include dialect, accent, prosody, vocabulary, speaking rate, vocal affect, vernacular, register (e.g., formality level), rhetorical style, and/or any other communication style.

Content can optionally be generated using one or more guardrails and/or one or more quality metrics. The guardrails and/or quality metrics can be predetermined, defined by a user, defined by a set of rules, defined by a model, and/or otherwise defined. The guardrails can function to ensure the generated content stays within its intended scope, constrain the generated content during generation (e.g., generation by a LLM), define hard requirements for content, and/or guard against content that is excessively or inappropriately clinical in nature. The guardrails can be passed into the content generation models as part of the prompt, used to condition the model (e.g., in the prompt, as a set of biasing weights, as a LoRA, etc.), used to check the resultant content (e.g., wherein a subsequent model evaluates whether the generated content violates the guardrail, etc.), and/or otherwise used. The guardrails can be a set of rules, a set of weights (e.g., a vector), a set of tools, and/or otherwise defined.

Guardrail verification of the content can be performed one or more times during S500. In an example, guardrail verification can include verifying a set of content with the guardrails after it is generated, determining a violation of the guardrails, repeating generation to produce a second set of content for the cohort, verifying the second set of content, and/or otherwise performing guardrail verification.

The guardrails are preferably applied deterministically (e.g., verified with a set of rules, by a human, etc.), but can alternatively be applied probabilistically (e.g., through a prompt).

An evaluator can evaluate generated content for adherence to the set of guardrails (e.g., during generation, after generation, etc.). The evaluator can include a guard model (e.g., a critic model, an evaluator).

Examples of guardrails that can be used include: compliance, content, writing, brand, operational constraints, and/or any other user-defined constraints. The compliance guardrails can include privacy (e.g., HIPAA compliance), FDA regulations (e.g., use of specific words; for example, "diabetes supplements" rather than "diabetes treatment"), using content with human approval, for marketing purposes, not clinical advice, and/or any other compliance guardrail. The content guardrails can include clinical guardrails (e.g., cannot provide medical diagnosis advice, cannot prescribe medications or treatments, cannot mention or prescribe any medical prescriptions, must exclude clinical recommendations or prescriptions, cannot give clinical recommendation, etc.), safety (e.g., harm detection and prevention, edge cases for problematic outputs, clinical safety validation, etc.), using general health language rather than specific medical terms, and/or any other content guardrail. The brand guardrails can include brand guideline compliance, brand tone, brand value propositions, and/or other guardrails. The operational constraint guardrails can include do-not-call list verification, resource limitations, and/or any other operational constraint.

Examples of quality metrics (e.g., measured quality metrics) for the generated content include: inclusive, accessible, clear, understandable, personalized and relevant to the cohort, safe, credible, evidence-based, actionable, and/or any other targets. Each quality metric can be scored (e.g., on a 5-point scale) based on criteria corresponding to the quality metric.

Content can be generated using one or more models (e.g., one or more large language models, vision language models, etc.).

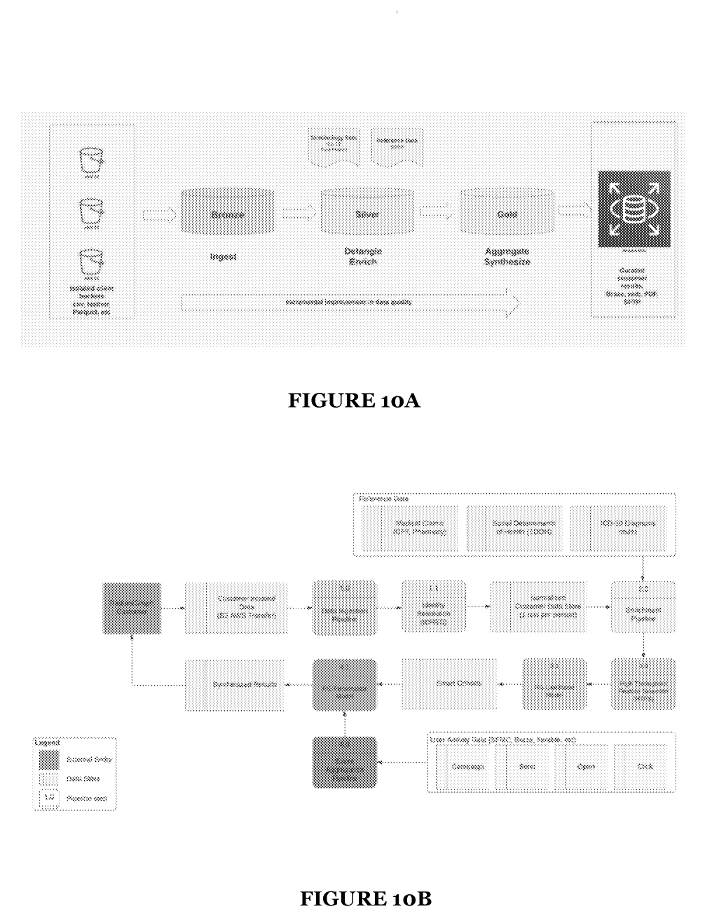

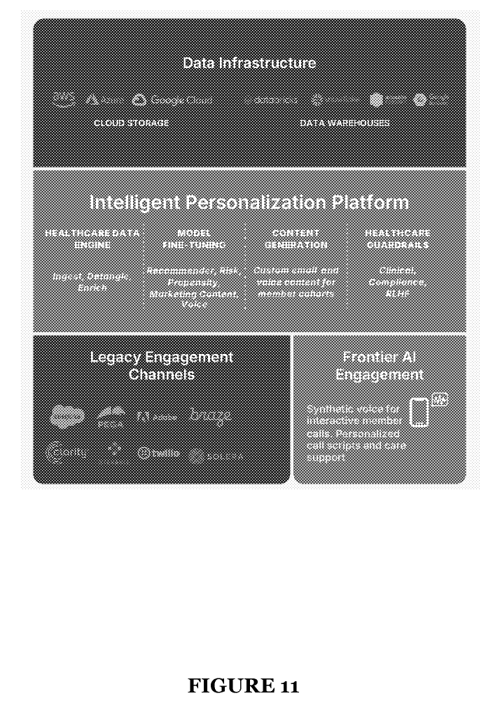

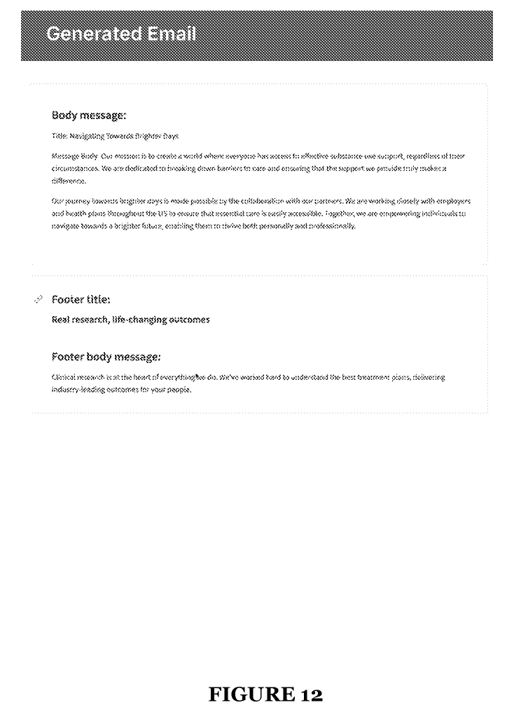

Examples are shown in FIG. 8B and FIG. 9.

In an example, the content can be generated using an actor model, a guardrail model, a critic model, a guidelines model, and/or any other model. In a specific example, the actor model can output generated content based on cohort information (e.g., the cohort description, other member data, etc.), guidelines (e.g., provided by a user, provided by a guidelines model, predetermined guidelines, all or a subset of the guardrails, etc.), one or more prompts, and/or any other suitable inputs.

In a specific example, the generated content can be passed to a guardrail model, which can output flags (e.g., flagging that the content does not pass one or more guardrail criteria). In a specific example, the generated content can be passed to a critic model, which can output evaluation metrics. The evaluation metrics can optionally be passed back to the actor model (e.g., via a guidelines model) to tune the generated content. During model training, a human can optionally evaluate generated content to provide feedback to train the one or more models.

An example is shown in FIG. 8A.

The generated content from S500 can be provided to the members, displayed to a user via a user interface 700, evaluated by subsequent models (e.g., guardrail models, verifiers, etc.), and/or otherwise processed. In illustrative examples, the generated content can be provided via email, a notification to a user device, a voice call (e.g., using an AI voice agent), physical mail, and/or otherwise delivered to the cohort.

In a first variant, the generated content is included in an email, SMS, mailer, or other text-based communication.

In a second variant, the generated content can be used as a script for a call (e.g., for a human caller, for generating audio using a generative model).

In a third variant, the generated content can be used as a script and/or set of prompts for generating a video using a generative model.

However, the generated content can be otherwise used.

The generated content can be sent to a member (e.g., a recipient member) in response to validation by a guard model, according to a schedule, in response to a user input, and/or otherwise sent.

S500 can optionally include tracking an outcome (e.g., interactions, engagements, product utilization, etc.) of the recipient member receiving the content, but can alternatively not include tracking an outcome. In an example, the system can include updating a model (e.g., smart choice model, propensity model, content generation model, etc.) based on the tracked outcome.

However, content can be otherwise determined.

### 5. Specific Examples

Specific example 1. A method comprising: determining datasets for each of a set of members in a population; extracting a population feature set from the datasets, wherein the population feature set comprises a member feature set for each of the set of members; segmenting the set of members into a set of cohorts based on the population feature set; generating a set of cohort metrics for each cohort in the set of cohorts based on member features of the members within the respective cohort; and generating content for a cohort in the set of cohorts based on the respective set of cohort metrics, wherein the content is constrained by a set of guardrails during generation and evaluated by a guard model after generation for adherence to the set of guardrails.

Specific example 2. The method of specific example 1, further comprising: before determining the set of cohorts, selecting a cohort creation feature set comprising a subset of the population feature set, based on a correlation between the cohort creation feature and a target variable.

Specific example 3. The method of specific example 2, wherein determining the set of cohorts comprises iteratively: selecting a new combination of cohort creation features from the cohort creation feature set that cover a largest number of non-cohorted members from the set of members; creating a new cohort associated with the new combination of cohort creation features; and assigning non-cohorted members that satisfy the new combination of cohort creation features to the new cohort.

Specific example 4. The method of specific example 3, wherein selecting the new combination of cohort creation features is performed by a machine learning model.

Specific example 5. The method of specific example 1, wherein the datasets comprise International Classification of Diseases (ICD) codes.

Specific example 6. The method of specific example 1, further comprising determining a propensity score for a member responding to a content delivery mechanism, based on the features for each of the set of members.

Specific example 7. The method of specific example 6, further comprising generating a content delivery strategy based on the propensity score.

Specific example 8. The method of specific example 1, further comprising, based on the set of cohort metrics, generating a cohort strategy comprising a behavioral strategy.

Specific example 9. The method of specific example 1, wherein the set of cohort metrics for a cohort comprises metrics derived from features outside of a combination of population features used to define the respective cohort.

Specific example 10. A method comprising: determining a dataset for a set of members; determining a set of features from the datasets for the set of members; based on the set of features, segmenting the set of members into a set of cohorts, comprising a) selecting a first combination of cohort creation features from the set of features that cover a largest number of non-cohorted members from the set of members; b) creating a new cohort associated with the first combination of cohort creation features; c) assigning a set of non-cohorted members that satisfy the new combination of cohort features to the new cohort; and d) repeating a)-c) until a stop condition is met; generating a description for each cohort in the set of cohorts comprising a set of cohort metrics; and generating content for a cohort based on the description.

Specific example 11. The method of specific example 10, further comprising enriching the dataset by associating social determinants of health data with members within the set of members.

Specific example 12. The method of specific example 10, further comprising enriching the dataset by associating International Classification of Disease codes with respective descriptions, wherein the set of features are extracted from the descriptions.

Specific example 13. The method of specific example 10, further comprising predicting a content strategy for a cohort, wherein the content strategy comprises a communication style, and wherein the content is further generated based on the content strategy.

Specific example 14. The method of specific example 10, wherein the content comprises a video generated by a generative model.

Specific example 15. The method of specific example 10, further comprising determining a content strategy for the cohort based on social determinants of health, wherein the content for the cohort is further generated based on the content strategy.

Specific example 16. The method of specific example 10, wherein generating content comprises aggregating content assets previously validated for adherence to guardrails by human evaluators, and excludes generating new content.

Specific example 17. The method of specific example 10, wherein the set of guardrails define hard requirements for content, comprising exclusions of any medical prescriptions.

Specific example 18. The method of specific example 10, wherein the content is generated based on a set of guardrails, the method further comprising: evaluating the content using a guard model configured to verify content compliance with a set of guardrails; and sending the content to a recipient member in response to validation by the guard model.

Specific example 19. The method of specific example 18, further comprising tracking an outcome of the recipient member receiving the content and updating a smart choice model based on the outcome.

Specific example 20. The method of specific example 19, wherein the smart choice model generates content on a per-member basis based on a predicted outcome.

All references cited herein are incorporated by reference in their entirety, except to the extent that the incorporated material is inconsistent with the express disclosure herein, in which case the language in this disclosure controls.

As used herein, "substantially" or other words of approximation can be within a predetermined error threshold or tolerance of a metric, component, or other reference, and/or be otherwise interpreted.

Optional elements, which can be included in some variants but not others, are indicated by broken lines in the figures. However, unbroken lines in the figures should not be interpreted to indicate that the depicted elements are essential, nor to indicate that the depicted elements may not be omitted from variants of the invention.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method comprising:
determining datasets for each of a set of members in a population;
extracting a population feature set from the datasets, wherein the population feature set comprises a member feature set for each of the set of members;
segmenting the set of members into a set of cohorts based on the population feature set;
generating a set of cohort metrics for each cohort in the set of cohorts based on member features of the members within the respective cohort; and
generating content for a cohort in the set of cohorts based on the respective set of cohort metrics, wherein the content is constrained by a set of guardrails during generation and evaluated by a guard model after generation for adherence to the set of guardrails.

2. The method of claim 1, further comprising: before determining the set of cohorts, selecting a cohort creation feature set comprising a subset of the population feature set, based on a correlation between the cohort creation feature and a target variable.

3. The method of claim 2, wherein determining the set of cohorts comprises iteratively: selecting a new combination of cohort creation features from the cohort creation feature set that cover a largest number of non-cohorted members from the set of members; creating a new cohort associated with the new combination of cohort creation features; and assigning non-cohorted members that satisfy the new combination of cohort creation features to the new cohort.

4. The method of claim 3, wherein selecting the new combination of cohort creation features is performed by a machine learning model.

5. The method of claim 1, wherein the datasets comprise International Classification of Diseases (ICD) codes.

6. The method of claim 1, further comprising determining a propensity score for a member responding to a content delivery mechanism, based on the features for each of the set of members.

7. The method of claim 6, further comprising generating a content delivery strategy based on the propensity score.

8. The method of claim 1, further comprising, based on the set of cohort metrics, generating a cohort strategy comprising a behavioral strategy.

9. The method of claim 1, wherein the set of cohort metrics for a cohort comprises metrics derived from features outside of a combination of population features used to define the respective cohort.

10. A method comprising:
determining a dataset for a set of members;
determining a set of features from the datasets for the set of members;
based on the set of features, segmenting the set of members into a set of cohorts, comprising:
a) selecting a first combination of cohort creation features from the set of features that cover a largest number of non-cohorted members from the set of members;
b) creating a new cohort associated with the first combination of cohort creation features;
c) assigning a set of non-cohorted members that satisfy the new combination of cohort features to the new cohort;
and
d) repeating a)-c) until a stop condition is met;
generating a description for each cohort in the set of cohorts comprising a set of cohort metrics; and
generating content for a cohort based on the description.

11. The method of claim 10, further comprising enriching the dataset by associating social determinants of health data with members within the set of members.

12. The method of claim 10, further comprising enriching the dataset by associating International Classification of Disease codes with respective descriptions, wherein the set of features are extracted from the descriptions.

13. The method of claim 10, further comprising predicting a content strategy for a cohort, wherein the content strategy comprises a communication style, and wherein the content is further generated based on the content strategy.

14. The method of claim 10, wherein the content comprises a video generated by a generative model.

15. The method of claim 10, further comprising determining a content strategy for the cohort based on social determinants of health, wherein the content for the cohort is further generated based on the content strategy.

16. The method of claim 10, wherein generating content comprises aggregating content assets previously validated for adherence to guardrails by human evaluators, and excludes generating new content.

17. The method of claim 10, wherein the set of guardrails define hard requirements for content, comprising exclusions of any medical prescriptions.

18. The method of claim 10, wherein the content is generated based on a set of guardrails, the method further comprising:
evaluating the content using a guard model configured to verify content compliance with a set of guardrails; and
sending the content to a recipient member in response to validation by the guard model.

19. The method of claim 18, further comprising tracking an outcome of the recipient member receiving the content, and updating a smart choice model based on the outcome.

20. The method of claim 19, wherein the smart choice model generates content on a per-member basis based on a predicted outcome.
